(19) United States
(12) Patent Application Publication
(10) Pub. No.: US 2005/0208503 A1
(43) Pub. Date: Sep. 22, 2005

(54) Chemical ligation of nucleic acids

(76) Inventors: Handy Yowanto, Walnut, CA (US); Changjun Yu, Pasadena, CA (US)

Correspondence Address: DORSEY & WHITNEY LLP, 555 CALIFORNIA STREET, SUITE 1000, SUITE 1000, SAN FRANCISCO, CA, 94104, US

(21) Appl. No.: 10/803,166

(22) Filed: Mar. 16, 2004

(51) Int. Cl. C12Q 1/68; C07H 21/04

(52) U.S. Cl. 435/6; 536/24.3

(57) ABSTRACT

The invention relates to the field of nucleic acid analysis. More particularly, the invention relates to compositions and methods used for the detection of sequence variations or single nucleotide polymorphisms (SNPs) in a nucleic acid of interest.

### FIELD OF THE INVENTION

The invention relates to the field of nucleic acid analysis. More particularly, the invention relates to compositions and methods used for the detection of sequence variations or single nucleotide polymorphisms (SNPs) in a nucleic acid of interest.

### BACKGROUND OF THE INVENTION

The detection of specific nucleic acids is an important tool for diagnostic medicine and molecular biology research. Gene probe assays currently play roles in identifying infectious organisms such as bacteria and viruses, in probing the expression of normal and mutant genes and identifying mutant genes such as oncogenes, in typing tissue for compatibility preceding tissue transplantation, in matching tissue or blood samples for forensic medicine, and for exploring homology among genes from different species.

Gene probe assays are commonly used to analyze the relationship between genetic variation and phenotype by identifying polymorphic DNA markers, such as single nucleotide polymorphisms (SNPs). Some SNPs, particularly those in and around coding sequences, are likely to be the direct cause of therapeutically relevant phenotypic variants and/or disease predisposition. There are a number of well known polymorphisms that cause clinically important phenotypes; for example, the apoE2/3/4 variants are associated with different relative risk of Alzheimer's and other diseases (see Cordor et al., Science 261(1993).

Ideally, a gene probe assay for the detection of polymorphisms should be sensitive, specific and easily automatable (for a review, see Nickerson, Current Opinion in Biotechnology 4:48-51 (1993)). The requirement for sensitivity (i.e. low detection limits) has been greatly alleviated by the development of the polymerase chain reaction (PCR) and other amplification technologies which allow researchers to amplify exponentially a specific nucleic acid sequence before analysis (for a review, see Abramson et al., Current Opinion in Biotechnology, 4:41-47 (1993)). For example, multiplex PCR amplification of SNP loci with subsequent hybridization to oligonucleotide arrays has been shown to be an accurate and reliable method of simultaneously genotyping hundreds of SNPs; see Wang et al., Science, 280:1077 (1998); see also Schafer et al., Nature Biotechnology 16:33-39 (1998).

Specificity, in contrast, remains a problem in many currently available assays gene probe assays. The extent of molecular complementarity between probe and target defines the specificity of the interaction. Variations in probe composition, the concentrations of probes, of targets and of salts in the hybridization medium, in the reaction temperature, and in the length of the probe may alter or influence the specificity of the probe/target interaction.

It may be possible under some circumstances to distinguish targets with perfect complementarity from targets with mismatches, although this is generally very difficult using traditional technology, since small variations in the reaction conditions will alter the hybridization. New experimental techniques with the necessary specificity for mismatch detection with standard probes include probe digestion assays in which mismatches create sites for probe cleavage and DNA ligation assays where single point mismatches prevent ligation.

There are a variety of enzymatic and non-enzymatic methods available for detecting sequence variations. Examples of enzyme based methods to detect variations in nucleotide sequences include, but are not limited to, Invader™, oligonucleotide ligation assay (OLA) single base extension methods, allelic PCR, and competitive probe analysis (e.g. competitive sequencing by hybridization).

A number of non enzymatic or template mediated chemical ligation methods have been developed that can be used to detect sequence variations. These include chemical ligation methods that utilize coupling reagents, such as N-cyanoimidazole, cyanogen bromide, and 1-ethyl-3-(3-dimethylaminopropyl)-carbodiimide hydrochloride. See Metelev, V. G., et al., (1999) Nucleosides & Nucleotides, 18:2711; Luebke, K. J., and Dervan, P. B. (1989) J. Am. Chem. Soc., 111:8733; and Shabarova, Z. A., et al., Nucleic Acids Research, 19:4247. Other chemical ligation methods react a 5′-tosylate or 5′-iodo group with a 3′-phosphorothioate group, resulting in a DNA structure with a sulfur replacing one of the bridging phosphodiester oxygen atoms. See Gryanov, S. M., and Letsinger, R. L., (1993) Nucleic Acids Research, 21:1403; Xu, Y. and Kool, E. T. (1997) Tetrahedron Letters, 38:5595; and Xu, Y. and Kool, E. T., (1999) Nucleic Acids Research, 27:875. Some of the advantages of using non-enzymatic approaches for the detection of polymorphisms include lower sensitivity to non-natural DNA analog structures, ability to use RNA target sequences, lower cost and greater robustness under varied conditions.

PCT applications WO 95/15971, PCT/US96/09769, PCT/US97/09739, PCT US99/01705, WO96/40712 and WO98/20162, all of which are expressly incorporated by reference, describe novel compositions comprising nucleic acids containing electron transfer moieties, including electrodes, which allow for novel detection methods of nucleic acid hybridization.

Accordingly, it is an object of the present invention to provide non-enzymatic methods in combination with electrochemical detection for detecting single nucleotide polymorphisms in nucleic acid sequences of interest.

### SUMMARY OF THE INVENTION

In accordance with the objectives outlined above, the present invention provides compositions and methods for detecting sequence variations in nucleic acid sequences of interest. The sequence variation may include a single base, i.e., a single nucleotide polymorphism, or several bases. The compositions and methods employ sequence-specific probes that permit the use of non-enzymatic methods of chemical ligation for the detection of sequence variations in a nucleic acid of interest. By using ligation probes that are labeled at either the 5′ end with an iodide moiety or at the 3′ end with a sulfur moiety, and that are either complementary over their entire length or differ in at least one base, nucleic acids comprising sequence variations can be detected based on whether or not a ligation product is formed. Additionally, one or more of the probes can comprise electron transfer moieties (ETMs), the presence of which is used as an indication of the presence of a nucleic acid comprising a sequence variation of interest.

The compositions of the present invention can further comprise a target nucleic acid comprising a first domain capable of binding in a sequence-specific manner to a first ligation probe, a second domain capable of binding in a sequence-specific manner to a second ligation probe, one of which can comprise at least one electron transfer moiety (ETM), and a detection position for which sequence information is desired. Either the first or second ligation probe includes an interrogation position having a base capable of binding in a sequence-specific manner to the base or bases located at the detection position on the target nucleic acid strand.

Preferably, the compositions of the present invention comprise one, two, three, and/or four ligation probes comprising interrogation positions and at least one electron transfer moiety with a distinguishable redox potential. For example, one of the four ligation probes may include a first base and an ETM with a first redox potential, the second ligation probe may include a second base and an ETM with a second redox potential, the third ligation probe may include a third base and an ETM with a third redox potential, and the fourth ligation probe may include a fourth base and an ETM with a fourth redox potential. Although any number of ETMs may be used in the compositions of the present invention, in preferred embodiments, the ETMs are ferrocene or ferrocene derivatives having redox potentials that are readily distinguishable using the methods described herein.

According to the invention, a target nucleic acid comprising a first domain capable of binding in a sequence-specific manner to a first ligation probe, a second domain capable of binding in a sequence-specific manner to a second ligation probe, and a detection position is contacted with a first ligation probe, and at least a second ligation probe comprising an interrogation position and an ETM with a distinguishable redox potential. If the base present at the interrogation position is capable of pairing in a sequence-specific manner with the base present at the detection position, a ligation complex comprising the target strand and a ligated strand comprising the first and second ligation probe is formed.

The identity of the base located at the interrogation position, and thus, the base present at the detection position in the target sequence can be determined by denaturing the ligation complex and adding the ligated strand to an electrode comprising a capture probe and a self-assembled monolayer and detecting electron transfer between the ETM present on the ligated strand and the electrode.

Alternatively, a target nucleic acid comprising a first domain capable of binding in a sequence-specific manner to a first ligation probe, a second domain capable of binding in a sequence-specific manner to a second ligation probe, and a detection position is contacted with a first ligation probe, and at least a second ligation probe comprising an interrogation position. In this embodiment, the identity of the base located at the interrogation position can be determined by denaturing the ligation complex and adding the ligated strand to an electrode comprising a capture probe, a self-assembled monolayer, and a label probe comprising a first domain that is capable of binding in a sequence-specific manner the ligated strand and a second domain comprising at least one ETM. Electron transfer between the ETMs present on the label probe and the electrode is an indication of the presence of the ligated strand comprising the base or sequence of interest.

In other embodiments, one ligation probe can be attached to an electrode using a capture probe. The attached ligation probe is contacted with one or more additional ligation probes comprising one or more ETMs and a target nucleic acid comprising a detection position. If the base located at the interrogation position on one of the ligation probes is capable of pairing in a sequence-specific manner with the base located at the detection position on the target nucleic acid, a ligation complex comprising the target nucleic acid and a ligated strand comprising the attached ligation probe and a second ligation probe is formed. The ligation complex is denatured and the base located at the detection position is identified by detecting electron transfer between the ETM present on the ligated strand and the electrode.

Other configurations also are possible for detecting a base or sequence of interest. For example, rather than attaching the ETMs to the ligation probes, the ETMs may be attached to a label probe. Following denaturation of the ligation complex, the ligated strand can be contacted with the label probe and detection of electron transfer between the ETMs on the label probe and the electrode used as an indication of the presence of the base of interest.

The compositions and methods of the present invention can be used in a variety of contexts. In a preferred embodiment, the compositions and methods of the present invention may be used to detect SNPs in a single nucleic acid or in a plurality of nucleic acids. In the latter embodiment, arrays of electrodes, each comprising a ligation probe with a different interrogation position are used.

### BRIEF DESCRIPTION OF THE DRAWINGS

FIGS. 1A and 1B illustrate the use of ligation probes to identify a nucleotide at a detection position within a target sequence. FIG. 1A illustrates the formation of a ligated strand when there are no mismatched nucleotides. In FIG. 1A, a target sequence 5 hybridized to a first 20 and a second 21 ligation probe. As outlined herein, either the first 20 or second 21 ligation probe comprises an electron transfer moiety 3 and a nucleotide at the interrogation 15 position that will hybridize to a nucleotide at the detection 10 position of the target sequence 5. If there are no mismatched nucleotide(s) present, a ligated strand 6 comprising the first and second ligation probe will be formed. FIG. 1B illustrates the situation when a mismatched nucleotide(s) 16 is present at the interrogation position. In this case, a ligated strand is not formed.

FIGS. 2A and 2B depict two embodiments of the present invention. FIG. 2A illustrates the use of a first ligation probe 20 comprising a 5′-iodide moiety, and a second ligation probe 21 comprising a 3′-sulfur moiety and an ETM 3. FIG. 2B illustrates the use of a first ligation probe 20 comprising a 5′-iodide moiety and a second ligation probe 21 comprising a 3′-sulfur moiety and a label sequence 2.

FIGS. 3A through 3E depict the formation of a ligation complex comprising a target sequence 5 and one, two, three and/or four ligation probes 21, 22, 23 and 24, each containing a different label 3, 7, 8, and 9 and a different nucleotide N_{A}, N_{B}, N_{C}, and N_{D} at the interrogation 15 position.

FIG. 4 depicts non enzymatic chemical ligation using a 3′-phosphothioate and 5′-iodo group.

FIGS. 5A and 5B depict activation of the terminal 5′-phosphate with N-cyanoimidazole or EDC.

FIG. 6 depicts attachment of multiple ETMs to a portion of a ligation probe. FIG. 6 illustrates a ligation probe 20 comprising a first portion 27 that hybridizes to a portion of the target sequence 5 and a second portion 28 comprising ETMs 3.

FIG. 7 depicts the use of a capture sequence to identify the base at the detection position. Target sequence 5 with detection position 10 is added to first ligation probe 20 with interrogation position 15 and capture sequence 30 and a second ligation probe 21 with a label 35 (although a label sequence could also be used, or, in the case of reporterless sensing, no label or label probe). If the interrogation base 15 is complementary to the detection position 10, ligation will occur, the ligated complex is denatured, and the ligated sequence is added to an array comprising an electrode 1 with a SAM 50 and capture probe 40 attached via an attachment linker 45. As described herein, other detection arrays can also be used.

FIGS. 8A-8C depict three preferred embodiments of the invention. In FIG. 8A, target sequence 5 with detection position 10 is added to first ligation probe 20 with interrogation position 15 and a second ligation probe 21. If the interrogation base 15 is complementary to the detection position 10, ligation will occur, the ligated complex is denatured, unreacted probes can be optionally removed and the ligated sequence is added to an array comprising an electrode 1 with a SAM 50 and capture probe 40 attached via an attachment linker 45. A label probe 55 with labels 35 (although again, these may not be required in some embodiments) is then added, which has a first portion complementary to a domain of the ligated probe. As described herein, other detection arrays can also be used. FIGS. 8B and 8C depict situations wherein one of the ligation probes serves as the capture probe. FIG. 8B shows the situation where one of the ligation probes has a label. FIG. 8C utilizes a label probe 55, with a first portion 60 that hybridizes to the ligated probe and a recruitment linker 75 with labels 35.

FIG. 9 depicts electron transfer moieties with different redox potentials that may be used as labels in the methods of the present invention.

FIG. 10 depicts the starting materials and autoligation using EDC described in Example 1.

FIGS. 11A-D depict ligation efficiency using various incubation times.

FIG. 12 depicts electrochemical detection of SNPs.

### DETAILED DESCRIPTION OF THE INVENTION

The present invention is directed to compositions and methods of determining the sequence of a target nucleic acid, using electrochemical detection on an electrode. The invention preferably includes the identification of a single nucleotide in a target nucleic acid, i.e., the detection of single nucleotide polymorphisms (SNPs).

The invention utilizes a non enzymatic, reagent free method for the ligation of oligonucleotides. In this method, two oligonucleotide strands bound at adjacent sites on a complementary target strand, undergo autoligation by displacement of a 5′-end iodide moiety with a 3′-end sulfur moiety. This reaction occurs with substantial specificity as to allow the detection of single base mismatches at either side of the ligation junction, as well as a few nucleotides away from the ligation junction. Ligation does not occur if mismatches are present.

In general, ligation probes are incorporated into a ligation complex, comprising a target molecule and a label, such as an electron transfer moiety (ETM). Additionally, the ligation probes are labeled at the 3′-end with a sulfur containing moiety or at the 5′-end with an iodide moiety. If there are no base mismatches between the target and the ligation probes, the ligation reaction proceeds efficiently, yielding a ligated strand. The ligation complex is denatured and added to an electrode comprising a capture probe. The presence or absence of the ligated strand is detected by monitoring the passage of electrons between the ETM and the electrode.

The presence or absence of the ETMs are detected as described below and in U.S. Pat. Nos. 5,591,578; 5,824,473; 5,770,369; 5,705,348 and 5,780,234; U.S. Ser. Nos. 08/911,589; 09/135,183; 09/306,653; 09/134,058; 09/295,691; 09/238,351; 09/245,105; 09/338,726; and 09/626,096; and PCT applications WO98/20162; WO 00/16089; PCT US99/01705; PCT US99/01703; PCT US00/10903 and PCT US99/10104, all of which are expressly incorporated herein by reference.

Accordingly, the present invention provides compositions and methods for detecting the presence or absence of a target sequence. By “target sequence” herein is meant a nucleobase sequence on a nucleic acid sought to be detected. The target sequence may be a portion of a gene, a regulatory sequence, genomic DNA, cDNA, RNA including mRNA and rRNA, or a product of an amplification process using a nucleic acid provided by nature or by a synthetic process. As is outlined herein, the target sequence may be a target sequence from a sample, or a secondary target such as the product of a chemical ligation reaction outlined herein. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

Each target sequence comprises a region of unique nucleobase sequence that may be used to discriminate one target sequence from another target sequence. The nucleobase sequence may comprise a single base, two or more contiguous bases, or two or more non contiguous bases. “Nucleobase” means those naturally occurring and those synthetic nitrogenous, aromatic moieties commonly found in the nucleic acid arts. Examples of nucleobases include purines and pyrimidines, genetically encoded nucleobases, analogs of genetically encoded nucleobases, and purely synthetic nucleobases. Specific examples of genetically encoded bases include adenine, cytosine, guanine, thymine, and uracil. Specific examples of analogs of genetically encoded bases and synthetic bases include 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). 5-propynyl-uracil, 2-thio-5-propynyl-uracil. Other non-limiting examples of suitable nucleobases include those nucleobases illustrated in FIGS. 2(A) and 2(B) of U.S. Pat. No. 6,357,163, incorporated herein by reference in its entirety.

Nucleobases can be linked to other moieties to form nucleosides, nucleotides, and nucleoside/tide analogs. As used herein, “nucleoside” refers to a nucleobase linked to a pentose sugar. Pentose sugars include ribose, 2′-deoxyribose, 3′-deoxyribose, and 2′,3′-dideoxyribose. “Nucleotide” refers to compound comprising a nucleobase, a pentose sugar and a phosphate. Thus, as used herein a nucleotide refers to a phosphate ester of a nucleoside, e.g., a triphosphate. Nucleic acid analogs, including nucleoside and nucleotide analogs, are described below.

The target sequence further comprises different target domains; for example, a first target domain of the target sequence may hybridize to a first ligation probe and a second target domain may hybridize to a second ligation probe. As more fully outlined below, the target sequence may also have a domain that hybridizes to a capture probe. The target domains may be adjacent or separated by one or more nucleotides. Unless specified, the terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

The target sequence comprises a position for which sequence information is desired, generally referred to herein as the “detection position”. In a preferred embodiment, the detection position is a single nucleotide, although in some embodiments, it may comprise a plurality of nucleotides, either contiguous with each other or separated by one or more nucleotides. By “plurality” is meant two or more nucleotides.

By “nucleic acid” or “oligonucleotide” or grammatical equivalents herein is meant at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids (LNAs), Koshkin et al., J. Am. Chem. Soc. 120:13252-3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997, page 35. All of these references are hereby expressly incorporated by reference. The modifications of the ribose-phosphate backbone may be done to facilitate the addition of ETMs, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

Particularly preferred nucleic acid analogs are peptide nucleic acids (PNA), and peptide nucleic acid analogs. “Peptide Nucleic Acid” or “PNA” refers to nucleic acid analogs in which the nucleobases are attached to a polyamide backbone through a suitable linker (i.e. methylene carbonyl, aza nitrogen) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. PNA backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This is particularly advantageous in the systems of the present invention, as a reduced salt hybridization solution has a lower Faradaic current than a physiological salt solution (in the range of 150 mM).

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, rather than target sequences, as this reduces non-specific hybridization, as is generally described in U.S. Pat. No. 5,681,702. Other preferred embodiments utilize diaminopurines (see e.g., Haaima et al., 1997, Nucleic Acids Res., 25: 4639-4643; and Lohse et al., 1999, Proc. Natl. Acad. Sci. USA, 96: 11804-11808).

The nucleic acid comprising the target sequence may be provided from any source. For example, the nucleic acid to be analyzed may be isolated or enriched from a sample, or be present in a cell or a tissue. As will be appreciated by those skilled in the art, in addition to the nucleic acid, the sample may comprise any number of other things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR amplification reaction); purified samples, such as purified genomic DNA, RNA, proteins, etc.; raw samples (bacteria, virus, genomic DNA, etc). As will be appreciated by those in the art, virtually any experimental manipulation may have been done on the sample.

If required, the target sequence is prepared using known techniques. As will be appreciated by those skilled in the art, the sample may be treated to lyse the cells, using known lysis buffers, electroporation, etc., with purification and/or amplification as needed. Examples of amplification processes that can be the source for the target sequence include, but are not limited to, Polymerase Chain Reaction (PCR), Ligase Chain Reaction (LCR), Strand Displacement Amplification (SDA; see, e.g., Walker et al., 1989, PNAS 89:392-396; Walker et al., 1992, Nucl. Acids Res. 20(7):1691-1696; Nadeau et al., 1999, Anal. Biochem. 276(2):177-187; and U.S. Pat. Nos. 5,270,184, 5,422,252, 5,455,166 and 5,470,723), Transcription-Mediated Amplification (TMA), Q-beta replicase amplification (Q-beta), Rolling Circle Amplification (RCA), Lizardi, 1998, Nat. Genetics 19(3):225-232 and U.S. Pat. No. 5,854,033), Asymmetric PCR (Gyllensten and Erlich, 1988, PNAS, 85:7652-7656) or Asynchronous PCR (see, e.g., WO 01/94638).

In a preferred embodiment, a step in the methods of the invention include a step to produce an excess of one strand over the other. As will be appreciated by those in the art, a variety of methods can be used, including, but not limited to, asymmetric polymerase chain reaction (APCR), exonuclease methods and the capture of the non-target strand.

In a preferred embodiment, asymmetric polymerase chain reaction (APCR) is used to enhance the production of the single stranded nucleic acid fragment used as the target sequence for detection as outlined herein. Traditional APCR techniques produces a single stranded bias by using the primers in a ratio of 100 to 1, although a variety of ratios ranging from 10:1 to 100:1 can be used as well (see, e.g., Gyllensten and Erlich, 1988, PNAS, 85:7652-7656; WO 00/20476; the disclosures of which are incorporated herein by reference in their entirety).

In a preferred embodiment, a novel nested primer method is used to amplify the patient sample. In this embodiment, an enhancement of target production is achieved using a two step process: a first symmetric PCR step, using a 1:1 ratio of primers, followed by the addition (preferably to the same reaction) of a second APCR step, using a ratio of 50:1 (again, with ratios of from about 10:1 to over 100:1 being useful). Alternatively, these reactions may be done in two steps as well. This has been shown to result in a 3-6 fold increase over a one step APCR reaction.

In a preferred embodiment, the asymmetric amplification step is accomplished as described in WO 00/20476, the disclosure of which is incorporated herein by reference in its entirety.

Accordingly, the compositions and methods of the present invention are used to identify the nucleotide(s) at the detection position located on a target sequence using ligation probes. The ligation probes are designed to “bind” or “hybridize” to target sequences that are complementary, such that double-stranded hybrids are formed between the ligation probes and the target sequence. “Complementary refers to sequences that contain no mismatched base pairs, e.g., A and T, A and U, C and G. “Binding” or “hybridization” refers to the base-pairing interactions of one nucleic acid strand with another that results in the formation of a double-stranded structure, a triplex structure or a quaternary structure.

As will be appreciated by a person skilled in the art, the probes also can be designed to bind to sequences that are “substantially complementary”, “identical”, or “substantially identical”. Two single stranded nucleic acid molecules are said to be substantially complementary when the nucleobases of one strand, optimally aligned and compared with the nucleobases of the other strand differ by one mismatched base pair, i.e., A and C, A and G, T and C, T and G. Two single stranded nucleic acid molecules are said to be “identical” when the nucleobases of one strand, optimally aligned and compared with the nucleobases of the other strand are the same, i.e., A and A, C and C, G and G. “Substantially identical” refers to sequences that contain 1 base that is not identical, i.e., A and C.

Nucleic acid samples that do not exist in a single-stranded state in the region of the target sequence(s) are generally rendered single-stranded in such region(s) prior to detection or hybridization. Generally, nucleic acid samples will be rendered single-stranded in the region of the target sequence using heat denaturation. For polynucleotides obtained via amplification, methods suitable for generating single-stranded amplification products are preferred. Non-limiting examples of amplification processes suitable for generating single-stranded amplification product polynucleotides include, but are not limited to, T7 RNA polymerase run-off transcription, RCA, Asymmetric PCR (Bachmann et al., 1990, Nucleic Acid Res., 18,1309), and Asynchronous PCR (WO 01/94638). Commonly known methods for rendering regions of double-stranded polynucleotides single stranded, such as the use of PNA openers (U.S. Pat. No. 6,265,166), may also be used to generate single-stranded target sequences on a polynucleotide.

The probes can be virtually any nucleic acid or nucleic acid analog that is capable of binding to a target sequence in a sequence-specific manner. Thus, probes useful in the invention include, but are not limited to DNA, RNA, PNA, and LNA, or mixtures, such as, PNA linked to LNA, LNA linked to DNA, or LNA linked to RNA. In preferred embodiments, the probes are DNA.

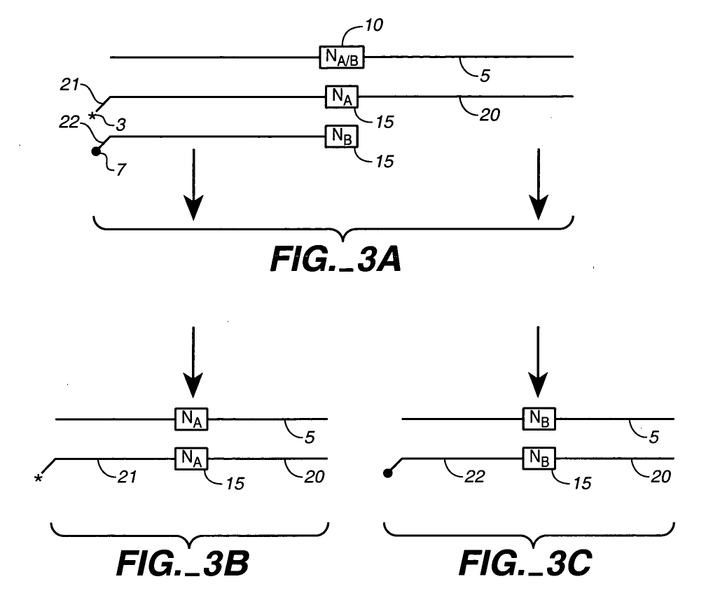

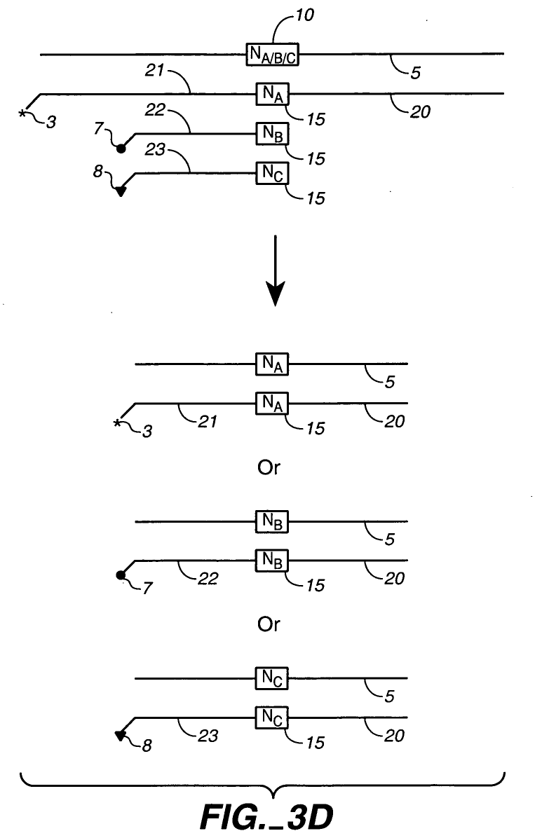

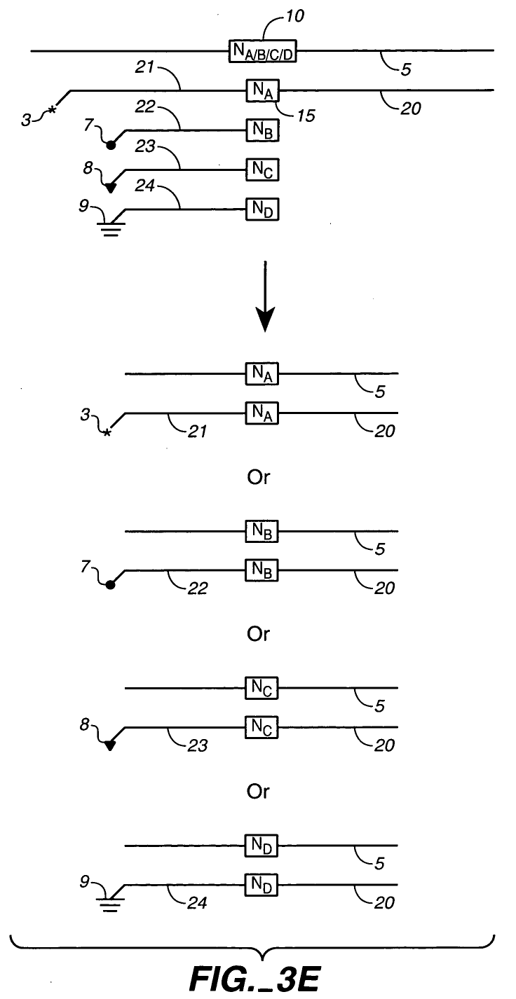

By “ligation probe” herein is meant a single stranded nucleic acid molecule that has a sequence that is complementary to a target specific sequence. A minimum of two ligation probes, a first and second ligation probe, are required to detect a single nucleotide polymorphism (SNP) or a sequence comprising two or more bases in a target sequence. In other embodiments, up to five ligation probes can be used, i.e. a first, second, third, fourth, and fifth ligation probes (see FIG. 3E). Unless specified, the terms “first”, “second”, “third”, “fourth” and “fifth” are not meant to confer an orientation as to which probe binds to which domain of the target sequence or which probe(s) have an interrogation position. In genotyping embodiments, at least one of the ligation probes comprises an interrogation position. By “interrogation position” herein is meant the base that base pairs with the detection position base in the target sequence.

The ligation probes comprise reactive groups to facilitate chemical ligation. Reactive groups include sulfur moieties, and leaving groups, such as iodide and tosylate (see, e.g., Xu and Kool, 1999, Nucleic Acids Research, 27(3): 875-881; the disclosure of which is incorporated herein by reference in its entirety). In a preferred embodiment, the leaving group is a 5′-iodide moiety. Preferably, modified nucleotides comprising a 5′ iodide moiety are incorporated during the solid phase synthesis of the ligation probe. See, for example, Xu and Kool, describing the solid phase synthesis of an oligonucleotide using a commercially available 5-iodothymidine phosphoramidite reagent (Glen Research; Xu, Y. and Kool, E. T., (1997) Tetrahedron Letters, 38:5595). A generalized structure for ligation probes comprising a 5′-iodide moiety is shown in structure 1:

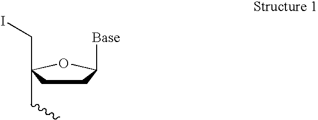

In a preferred embodiment one of the ligation probes comprise a 3′-sulfur moiety. The sulfur group can be incorporated into an oligonucleotide strand using a phosphorothioate group (see Herrlein, M. K., et al., (1997) J. Am. Chem. Soc., 117:10151; Xu, Y. and Kool, E. T., (1997) Tetrahedron Letters, 38:5595; the disclosures of which are incorporated herein by reference). Sulfur groups also can be incorporated into an oligonucleotide strand using dithiophosphate. A generalized structure for ligation probes comprising a 3′-sulfur moiety is shown in structure 2:

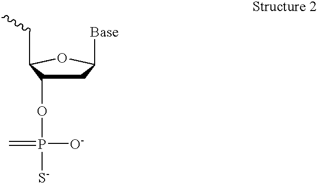

Unless specified, the terms “first”, “second”, “third”, “fourth” and “fifth” are not meant to confer an orientation of the sulfur and iodide moieties. For example, the first ligation probe may be labeled at its 5′ end with an iodide moiety or at its 3′end with a sulfur moiety. Similarly, the second ligation probe may be labeled at its 5′-end with an iodide moiety or at its 3′-end with a sulfur moiety. The same is true for any of the probes used in the methods of the invention.

In some embodiments, the 5′- and 3′ moieties are hydroxyls. In this embodiment, a ligated strand comprising a first and second ligation probe may be formed by activating the terminal 5′-phosphate in the presence of a coupling reagent. Suitable coupling reagents include N-cyanoimidazole (Kanaya, R. and Yanagawa, H., (1986) Biochemistry, 25:7423; Luebke, K. J. and Dervan, P. B., (1989) J. Am. Chem. Soc., 111:8733), 1-ethyl-3-(3-dimethylaminopropyl)carbodiimide hydrochloride (EDC) (Metelev, V. G., et al., (1999) Nucleosides & Nucleotides 18:2711), BrCN (Shabarova et al., 1991, Nucleic Acids Research, 19(15):4247-4251), and K₃Fe(CN)₆ or Kl₃ (Gryaznoz, S. M. and Letsinger, R. L., (1993) Nucleic Acids Research, 21:1403; the disclosures of which are incorporated herein by reference).

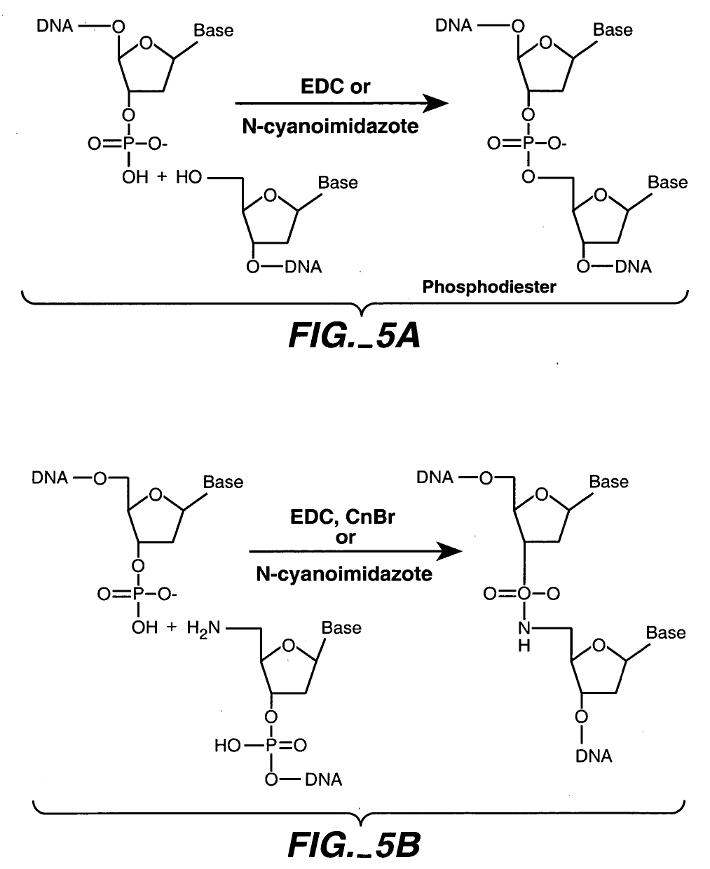

In a preferred embodiment, a terminal 5′-phosphate is activated in the presence of a coupling reagent. FIGS. 5A and 5B depict activation of the terminal 5′-phosphate with N-cyanoimidazole or EDC.

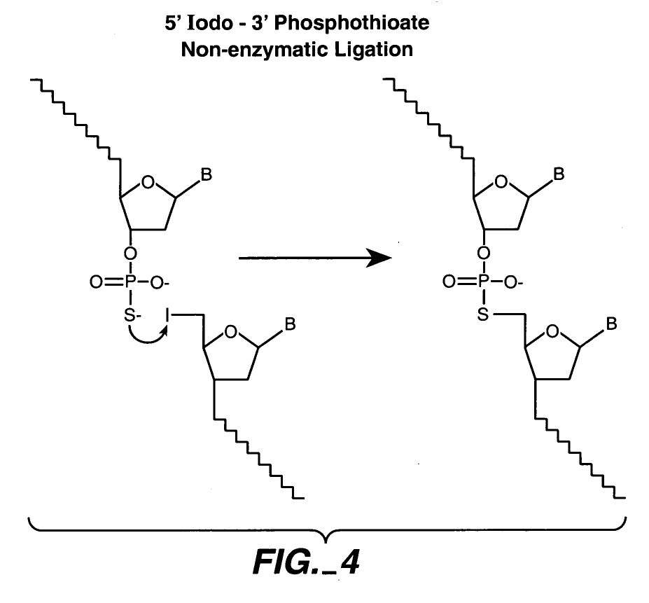

FIG. 4 depicts non enzymatic chemical ligation using a 3′-phosphothioate and 5′-iodo group. As shown in FIG. 4, the 3′-sulfur moiety displaces the 5′-iodide moiety resulting in a joined, i.e. ligated, nucleic acid strand. This reaction occurs without the need for added reagents. The resulting ligated strand differs from natural DNA by the replacement of a single oxygen atom with a single sulfur atom. As a result of the replacement of the single oxygen atom with a single sulfur atom, a bridging 5′-phosphorothioester is formed. The bridging 5′-phophorothioester is stable in solution, resistant to enzymatic hydrolysis and does not affect the ability of polymerases to replicate or transcribe the ligated strand (see, e.g., Xu and Kool, 1999, Nucleic Acids Research, 27(3):875-881).

One or more of the ligation probes can also include a detectable label. By “label” or “detectable label” herein refers to a moiety that, when attached to a probe of the invention, renders such a probe detectable using known detection methods, e.g., electronic, spectroscopic, photochemical, or electrochemiluminescent methods. Exemplary labels include, but are not limited to, electron transfer moieties (ETMs).

In a preferred embodiment, at least one of the ligation probes comprises a first ETM. By “ETM”, “electron donor moiety”, “electron acceptor moiety”, or grammatical equivalents herein refers to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred ETMs include, but are not limited to, transition metal complexes, organic ETMs, and electrodes. See, e.g., U.S. Pat. Nos. 6,096,273, 6,264,825, and 6,221,583 for a discussion of suitable ETMs, the disclosures of which are incorporated herein by reference.

In a preferred embodiment, the ETMs are transition metal complexes. Transition metals are those whose atoms have a partial or complete d shell of electrons. Suitable transition metals for use in the invention are described, for example, in U.S. Pat. Nos. 6,096,273, 6,264,825, and 6,221,583 the disclosures of which are incorporated herein by reference.

In addition to transition metal complexes, other organic electron donors and acceptors may be covalently attached to the nucleic acid for use in the invention. These organic molecules are described, for example, in U.S. Pat. Nos. 6,096,273, 6,264,825, and 6,221,583, the disclosures of which are incorporated herein by reference.

Preferred ETMs are metallocenes, with ferrocene and ferrocene derivatives being particularly preferred. See, e.g., U.S. Pat. Nos. 6,096,273, 6,264,825, and 6,221,583, and U.S. Publication No. 20030143556, the disclosures of which are incorporated herein by reference.

In one embodiment, the electron donors and acceptors are redox proteins as are known in the art. However, in some embodiments, redox proteins are not preferred.

In one embodiment, ETMs, such as Ru²⁺(bpy)₃, can be used for detection methods based on electrochemiluminscence (see Blackburn, 1991, Clin Chem, 37:1534-1539). However, in some embodiments, ETMs are not preferred for use in detection methods based on electrochemiluminscence.

The choice of the specific ETMs will be influenced by the nature of the assay, i.e., detection of SNPs with two or more alleles, and by the type of electron transfer detection used. For the detection of SNPs with two or more alleles, ETMs with different redox potentials are used (see U.S. Publication No. 20030143556, the disclosure of which is incorporated herein by reference). The redox potentials of the ETMs are chosen such that they are distinguishable in the assay system used. By “redox potential” (sometimes referred to as E₀) herein is meant the voltage which must be applied to an electrode (relative to a standard reference electrode such as a normal hydrogen electrode) such that the ratio of oxidized and reduced ETMs is one in the solution near the electrode. In a preferred embodiment, the redox potentials are separated by at least 100 mV, although differences either less than this or greater than this may also be used, depending on the sensitivity of the system, the electrochemical measuring technique used and the number of different labels used.

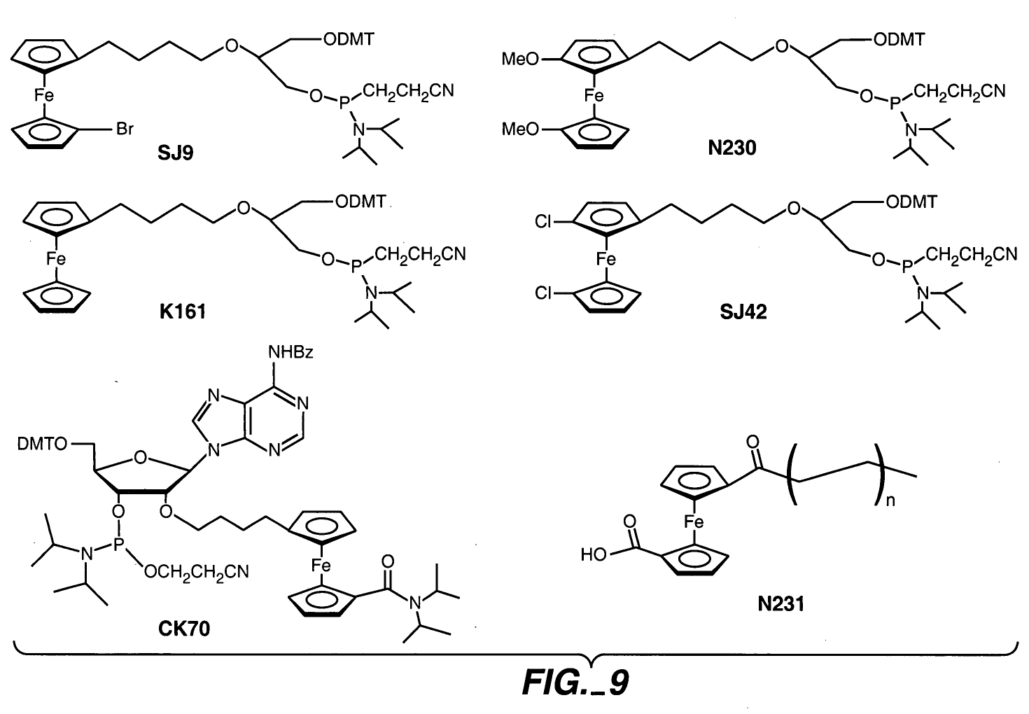

In a particularly preferred embodiment, derivatives of ferrocene are used; for example, ferrocenes with or without ring substituents, i.e., the addition of an amine or an amide, a carboxylate, a halogen, etc. FIG. 9 illustrates ferrocene derivatives with different redox potentials. See also U.S. Publication No. 20030143556 for a discussion of ETMs with distinguishable redox potentials, the disclosure of which is incorporated herein by reference in its entirety.

In a preferred embodiment, each ligation probe having an interrogation position with a different base, has a covalently attached ETM with a distinguishable redox potential.

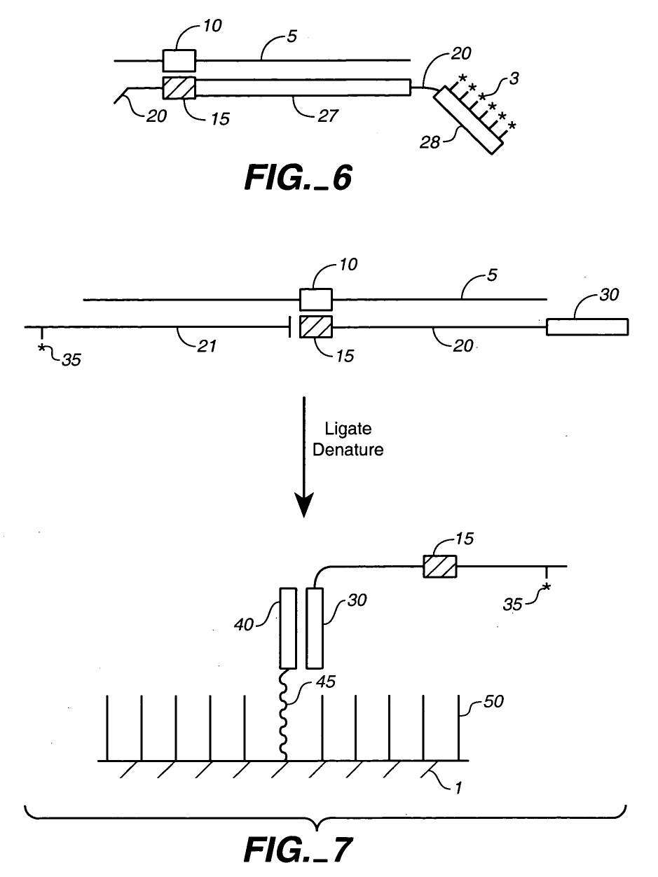

In a preferred embodiment, a plurality of ETMs are used on a single ligation probe. The use of multiple ETMs provides signal amplification, and thus, allows more sensitive detection limits. While the use of multiple ETMs on nucleic acids that hybridize to complementary strands can decrease the Tms of the hybridization complexes depending on the number, site of attachment and spacing between the multiple ETMs, this is not a factor when the ETMs are localized to a portion of the probe which does not hybridize to a complementary sequence (see FIG. 6). The portion of the probe that includes the ETMs, but lacks a complementary sequence for a target nucleic acid or another nucleic acid molecule used in the methods of the present invention is referred to herein as a “recruitment linker”.

In a preferred embodiment, multiple ETMs are used on a recruitment linker, since this linker does not hybridize to a complementary sequence. Preferably, the recruitment linker is nucleic acid, or nucleic acid analog. Pluralities of ETMs are preferred, with at least about 2 ETMs per recruitment linker being preferred, and about 8 being particularly preferred, and at least about 10 to 50 being preferred insome cases. In some instances, very large numbers of ETMs, i.e. 50 to 1000, can be used. In general, the number chosen depends on the system, the required sensitivity, and the solubility of the ETMS, etc. In the case of ferrocenes generally from 4-10 per probe are used, with 8 being particularly preferred.

The probes of the present invention may be synthesized using routine methods. For example, methods of synthesizing oligonucleotide probes are described in U.S. Pat. No. 4,973,679; Beaucage, 1992, Tetrahedron 48:2223-2311; U.S. Pat. No. 4,415,732; U.S. Pat. No. 4,458,066; U.S. Pat. No. 5,047,524 and U.S. Pat. No. 5,262,530; the disclosures of which are incorporated herein by reference. The synthesis may be accomplished using automated synthesizers available commercially, for example the Model 392, 394, 3948 and/or 3900 DNA/RNA synthesizers available from Applied Biosystems, Foster City, Calif.

Methods of synthesizing labeled probes with ETMS attached via: (1) a base; (2) the backbone, including the ribose, the phosphate, or comparable structures in nucleic acid analogs; (3) nucleoside replacement, or (4) metallocene polymers, are also well-known. As specific examples, see WO 00/20476 and U.S. Publication Nos. 20020121314 and 20030143556; the disclosures of which are incorporated herein by reference.

The basic principle of the chemical ligation assay for the detection of SNPs is illustrated in FIGS. 3A-3E. FIGS. 3A-C depict an embodiment for detection of a SNP that exists as two different alleles. In this embodiment, three probes are used. One of the three probes, i.e. 20, lacks an interrogation base and ETM, but includes a sequence that is complementary to one of the two domains of the target strand 5. The other two probes, 21 and 22, include an interrogation position 15 and are labeled with ETMs having two different redox potentials, redox potential 3 and redox potential 7. As shown in FIG. 3B, a ligated strand comprising ligation probes 20 and 21 and ETM 3 is formed if the base N_{A} at interrogation position 15 on probe 21 is complementary to the base N_{A} at the detection position 10 on the target sequence. If the base N_{A} at interrogation position 15 on probe 21 is not complementary to the base N_{B} at the detection position 10 on the target strand, a ligated strand comprising ligation probes 20 and 21 is not formed. However, as shown in FIG. 3C, a ligated strand comprising ligation probes 20 and 22 and ETM 7 is formed if the base N_{B} at interrogation position 15 on probe 22 is present. Detection of ETM 3 or 7 is used to identify which base is present at the detection position on the target strand 5.

FIG. 3D illustrates an embodiment for detecting a SNP with 3 alleles. In this embodiment, 4 ligation probes are used. A first probe 20, having a sequence complementary to the target strand 5, and three ligation probes, 21, 22, and 23, having a sequence complementary to a different domain of the target strand 5, an interrogation position 15, and ETMs with different redox potentials, 3, 7 and 8. A ligated strand, comprising ligation probe 20 and one of the other three probes 21, 22 and 23, will be formed depending on which probe has a complementary base at interrogation position 15 to the base present at detection position 10. For example, if the base N_{C} is present at the detection position, than a ligated strand comprising ligation probe 20 and 23 will be formed. Similarly if base N_{B} is present at the detection position, than a ligated strand comprising ligation probes 20 and 22 will be formed. Likewise, if base N_{A} is present at the detection position, than a ligated strand comprising ligation probes 20 and 21 will be formed.

FIG. 3E illustrates an embodiment for detecting a SNP with 4 alleles. In this embodiment, 5 ligation probes are used. A first probe 20, having a sequence complementary to the target strand 5, and three ligation probes, 21, 22, 23, and 24, having a sequence complementary to a different domain of the target strand 5, an interrogation position 15, and ETMs with different redox potentials, 3, 7 8, and 9. A ligated strand, comprising ligation probe 20 and one of the other four probes 21, 22, 23, and 24, will be formed depending on which probe has a complementary base at interrogation position 15 to the base present at detection position 10. For example, if the base N_{D} is present at the detection position, than a ligated strand comprising ligation probe 20 and 24 will be formed. If, on the other hand, the base N_{C} is present at the detection position, than a ligated strand comprising ligation probe 20 and 23 will be formed. Similarly if base N_{B} is present at the detection position, than a ligated strand comprising ligation probes 20 and 22 will be formed. Likewise, if base N_{A} is present at the detection position, than a ligated strand comprising ligation probes 20 and 21 will be formed. Thus, by using different probes, each with a different base at the interrogation position and each with a different label, the identification of the base at the detection position can be elucidated.

Accordingly, the ligation substrates of the invention can take on a number of configurations.

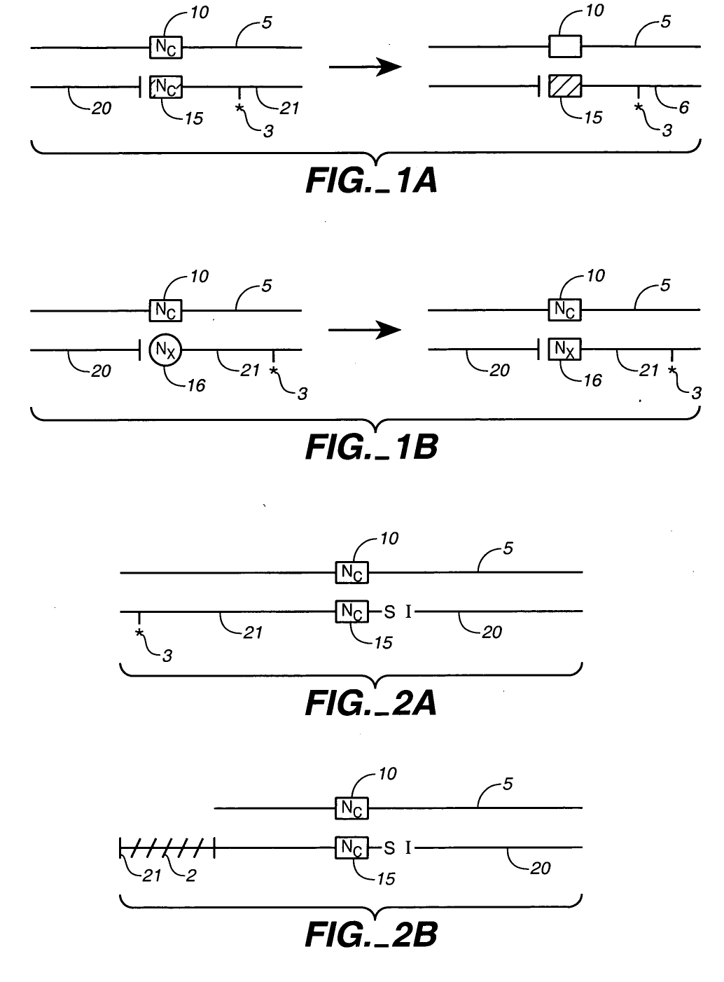

By “ligation substrate” herein is meant a substrate for chemical ligation comprising at least one target nucleic acid strand and two or more ligation probes. For example, as shown in FIG. 1A, the ligation substrate comprises target strand 5 and ligation probes 20 and 21. Once a ligation substrate is formed, and chemical ligation occurs, a “ligation complex” is formed, i.e., the structure depicted in FIG. 1A, comprising target strand 5 and ligated strand 6. As used herein, “ligated strand” refers to the nucleic acid strand formed by joining two ligation probes together using the methods of the present invention.

In a preferred embodiment, the ligation substrate comprises at least one target nucleic acid strand and two ligation probes, one of which comprises an interrogation position and either a label or a label sequence to which a label probe binds, as described below (see FIGS. 3A-3C). Alternatively, at least one target nucleic acid strand and three or more ligation probes are used (see FIGS. 3D and 3E). In this embodiment, the first ligation probe is complementary to the first domain of the target sequence, and the other ligation probes comprise different bases at the interrogation position, as well as different labels or different label sequences. Only when the correct ligation probes (e.g. with the perfectly complementary sequence) hybridize to the target nucleic acid strand will ligation occur.

As will be appreciated by a person skilled in the art, more than one detection position can be evaluated using ligation probes with different bases at the interrogation position and distinguishable labels. For example, in FIG. 3E, target nucleic acid strands with different bases at the detection position, i.e. a target strand with base N_{A}, a target strand with base N_{B}, a target strand with base N_{C} and a target strand with base N_{D} can be added to a ligation complex comprising several copies of ligation probes 20, 21, 22, 23 and 24. Formation of ligated strands can be detected based on the redox potentials of the ETMs, i.e., a ligated strand comprising ligation probes 20 and 21 and ETM 3, a ligated strand comprising ligation probes 20 and 22, and ETM 7, etc.

Accordingly, in a preferred embodiment, the ligation substrate comprises a target nucleic acid strand and two ligation probes. As is depicted in FIG. 2A, the first ligation probe can comprise an iodide moiety and the second ligation probe can comprise the interrogation position, the sulfur moiety, and a label (FIG. 2A) or a label sequence (FIG. 2B) to which a label probe will hybridize. Either probe may also comprise an optional capture sequence for subsequent hybridization to a complementary sequence present on a capture probe, as outlined below.

As is shown in FIG. 1A, the ligation substrate comprises a target nucleic acid strand with a first domain that hybridizes to a first ligation probe and a second domain that hybridizes to a second ligation probe. If the second ligation probe comprises an interrogation position with a base complementary to the base located at the detection position, i.e. (N_{C}), on the target strand, and the first ligation probe is substantially complementary to the first domain, a ligation complex is formed comprising the target sequence and a ligated strand consisting of the two ligation probes. If, however, as shown in FIG. 1B, the second ligation probe comprises an interrogation position with a base that is not complementary to the base located at the detection position, i.e. Nx, a ligated strand will not be formed.

In a preferred embodiment, the invention provides different ligation probes with different bases and different redox labels. This is analogous to the “two color” or “four color” idea of competitive hybridization and is also analogous to sequencing by hybridization. For example, sequencing by hybridization has been described (Drmanac et al., Genomics 4:114 (1989); Koster et al., Nature Biotechnology 14:1123 (1996); U.S. Pat. Nos. 5,525,464; 5,202,231 and 5,695,940, among others, all of which are hereby expressly incorporated by reference in their entirety).

For example, the methods of the present invention can use a ligation substrate comprising a target strand and three ligation probes. The first ligation probe 20 is substantially complementary to either the first or second domain of the target sequence. The second ligation probe 21 comprises an interrogation position with a first base and a first ETM. The third ligation probe 22 comprises an interrogation position with a second base and a second ETM. A ligation complex will be formed if either the second or third probe comprises an interrogation position with a base that is complementary to the base at the detection position on the target strand. Thus, a ligation complex comprising the target strand and the first and second ligation probes may be formed (FIG. 3B). Alternatively, a ligation complex comprising the target strand and the first and third ligation probes may be formed (FIG. 3C).

In other preferred embodiments, a ligation substrate comprising a target strand, a first ligation probe 20 substantially complementary to either the first or second domain of the target strand, second 21, third 22 and fourth 23 ligation probes comprising interrogation positions and ETMs is used (FIG. 3D). The second ligation probe comprises an interrogation position with a first base and a first ETM, the third ligation probe comprises an interrogation position with a second base and a second ETM, and the fourth ligation probe comprises an interrogation position with a third base and a third ETM. Thus, a ligation complex comprising the first ligation probe and either the second, third or fourth ligation probes is possible depending on which probe comprises an interrogation position with a base that is complementary to the base at the detection position on the target strand (FIG. 3D).

In a preferred embodiment, a ligation substrate comprising a target strand, a first ligation probe 20 substantially complementary to either the first or second domain of the target strand, second 21, third 22, fourth 23, and fifth 24 ligation probes comprise interrogation positions and ETMs is used (FIG. 3E). The second ligation probe comprises an interrogation position with a first base and a first ETM, the third ligation probe comprises an interrogation position with a second base and a second ETM, the fourth ligation probe comprises an interrogation position with a third base and a third ETM, and the fifth ligation probe comprises an interrogation position with a fourth base and a fourth ETM. Thus, a ligation complex comprising the first ligation probe and either the second, third, fourth, or fifth ligation probe is possible depending on which probe comprises an interrogation position with a base that is complementary to the base at the detection position on the target strand.

Accordingly, the ligation probes are hybridized to the target sequence to form a ligation complex. This method is based on the fact that two oligonucleotides hybridized at adjacent sites on a target strand undergo chemical ligation by displacement of a 5′-end iodide moiety with a 3″-end sulfur moiety, if complementarity exists at the two bases being ligated together. Thus, in this embodiment, the target sequence comprises a contiguous first target domain adjacent to a second target domain comprising the detection position. That is, the detection position is “between” the first target domain and the rest of the second target domain. A first ligation probe is hybridized to the first target domain and a second ligation probe is hybridized to the second target domain. If the second ligation probe has a base complementary to the detection position base, and the adjacent base on the first probe is complementary to the corresponding base on the target strand, chemical ligation will proceed resulting in the ligation of the first and second ligation probes such that a ligated strand or probe is formed. If this complementarity does not exist, a ligated strand is not formed.

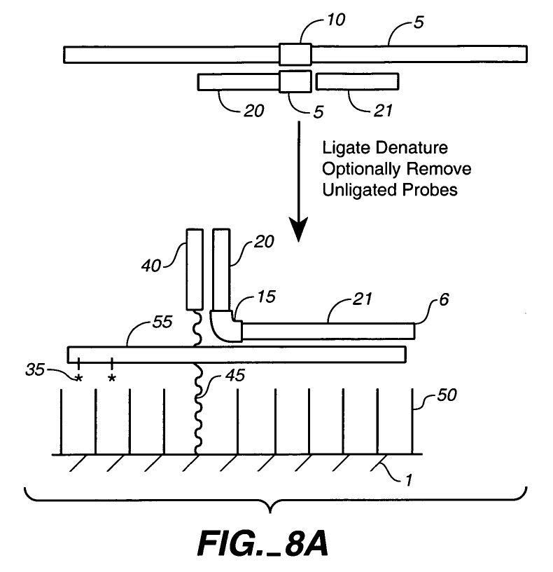

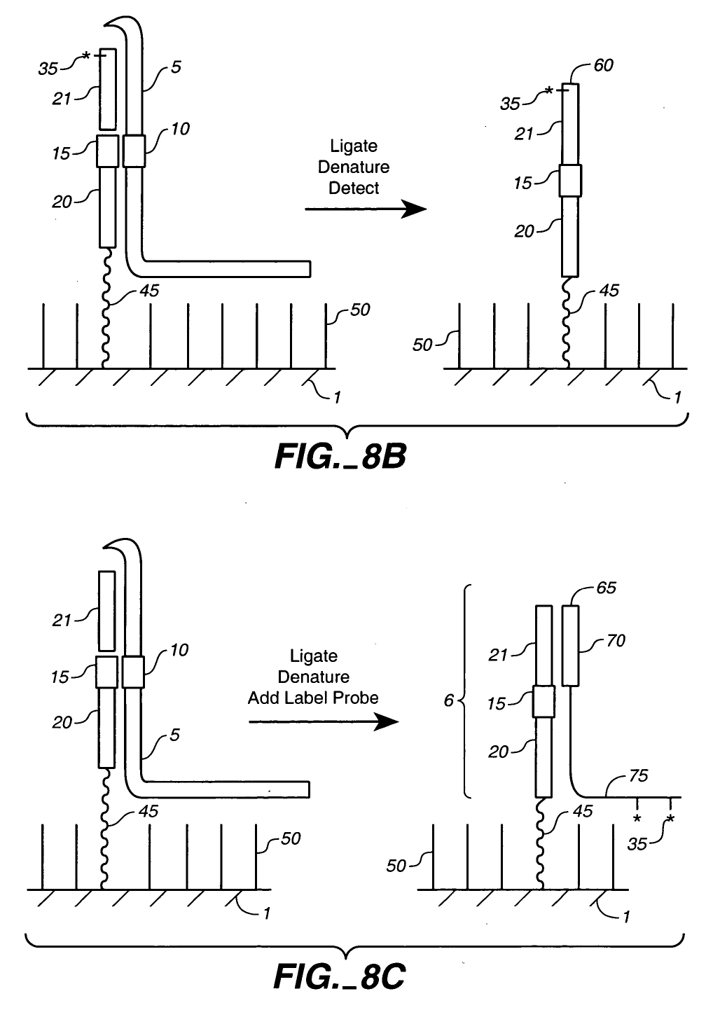

The ligation substrate can be unbound, or bound to a solid support. For example, the target strand, and all of the ligation probes can be added to a suitable ligation buffer and incubated for a period of time sufficient for the formation of a ligation complex (see FIG. 8A). Alternatively, a component of the ligation substrate may be attached to a solid support. For example, as illustrated in FIG. 8B or 8C, one of the ligation probes can be attached to an electrode via an attachment linker, and the other ligation probe and target strand free in solution.

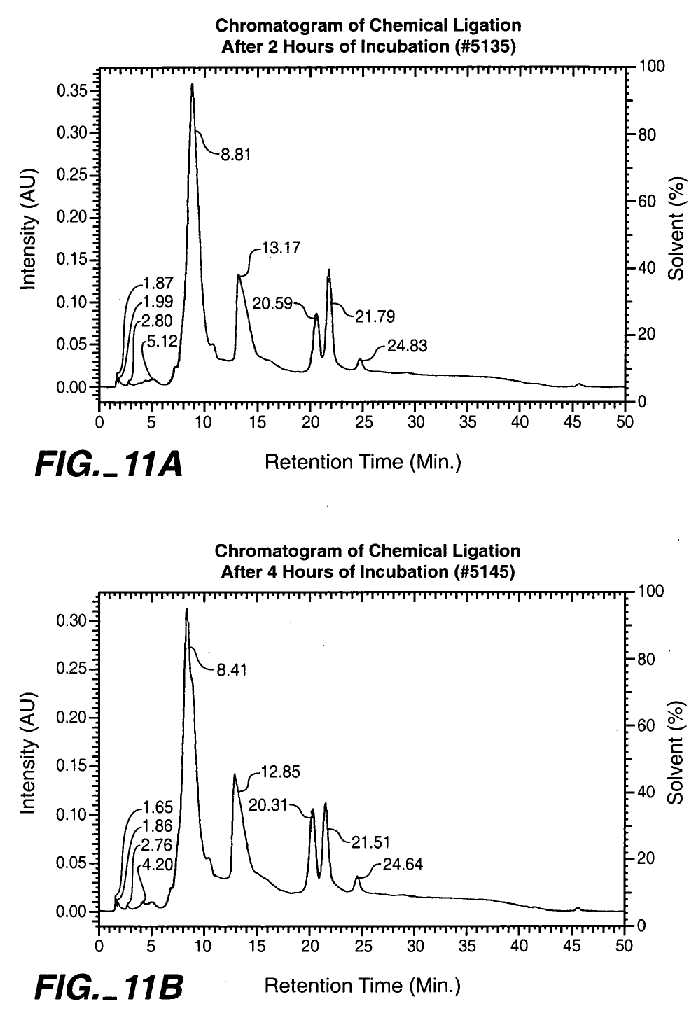

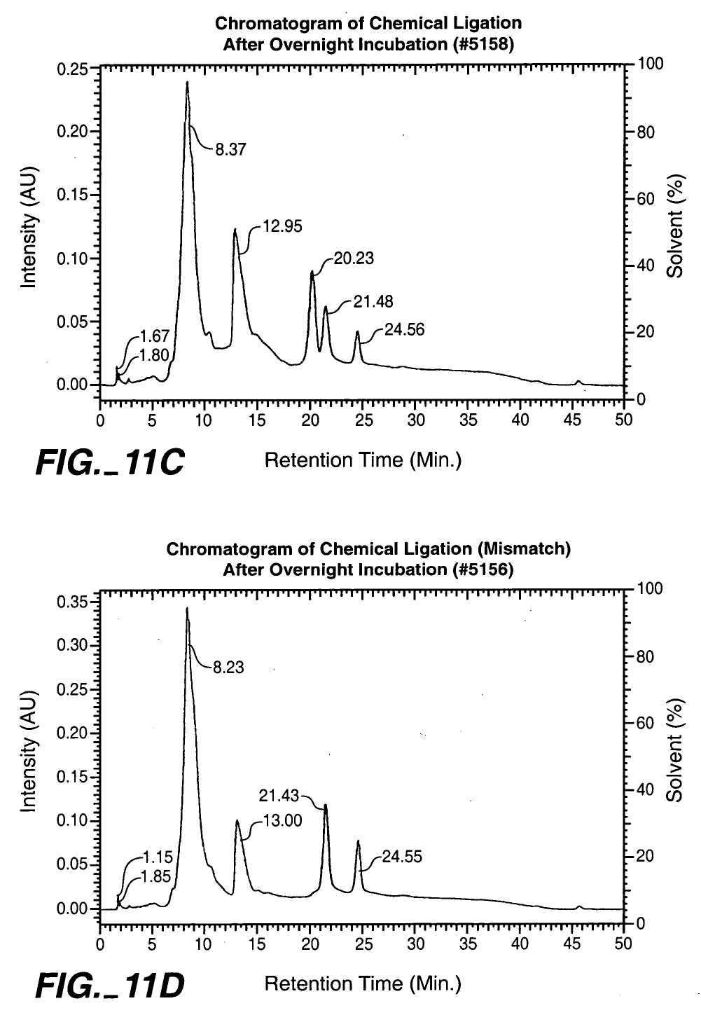

The efficiency of ligation will vary depending on whether a polymorphism, i.e., mismatch occurs at the detection position. A ligation complex with no mismatch at the detection position (i.e. “matched complex”) can be distinguished from a mismatched ligation complex by increasing the temperature, as a “matched” ligation complex is more stable than a complex comprising a mismatch. Depending on the location of the mismatch, (e.g. proximity to the ligation junction) and the type of probe (e.g. padlock probe) temperatures between 0° C. to 70° C. are used to distinguish mismatched ligation complexes from matched ligation complexes. See Xu, Y. and Kool, E. T., (1999) Nucleic Acids Research, 27:875; Metelev, V. G., et al., (1999) Nucleosides & Nucleotides, 18:2711; and FIGS. 11A-D, and 12.

Conditions for efficient ligation are known to those of skill in the art (see, e.g., Xu and Kool, 1997, Tetrahedron Letters, 38(32):5595-5598. Variables that may be varied to optimize ligation conditions include location of the mismatch temperature, incubation time, target/probe concentrations, salt concentration, pH, as well as other components of the ligation buffer. For example, temperatures between 16° C. and 23° C. may be used, and ligation reactions may be incubated between 2 to 20 hours.

In some embodiments, heat cycling is used to allow the ligated strand to be denatured off the target sequence such that it may serve as a template for further reactions. In these embodiments, temperatures between 92° C. to 95° C. are used to denature the ligated strand/target strand complex.

In a preferred embodiment, the ligated strand is disassociated from the target sequence using heat denaturation and added to an assay complex. By “assay complex” herein is meant the collection of hybridization complexes comprising nucleic acids that contain at least one ETM and thus allow detection. The composition of the assay complex depends on the use of the different probe components outlined herein. For example, as illustrated in FIG. 8A, the assay complex can comprise a capture probe 40 attached to an electrode 1 via an attachment linker 45, a label probe 55 comprising one or more ETMs 35, and a ligated strand 6 comprising ligation probes 20 and 21.

In other embodiments, such as the embodiment illustrated in FIG. 8B, the assay complex can comprise a ligation probe 20 comprising an interrogation position 15 attached to an electrode 1 via an attachment linker 45 to which is added a target strand 5 having a detection position 15. A second ligation probe 21 with an ETM 35 can then be added. If the base at the interrogation position is complementary to the base present at the detection position, a ligated strand will be formed that can be detected following disassociation of the target strand.

In yet other embodiments, such as illustrated in FIG. 8C, the assay complex can comprise a ligated strand 6 and a label probe 55 comprising a first portion that hybridizes to the ligated strand 6 and a second portion comprising a recruitment linker 75 and ETMs 35.

The assays are generally run under stringency conditions which allows formation of an assay complex only in the presence of a substantially complementary capture probe. Alternately, the hybridization complex can comprise the target sequence, a two ligation probes, one of which is attached to an electrode via an attachment linker. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions; for example, when an initial hybridization step is done between the ligated strand and the capture probe. Running this step at conditions which favor specific binding can allow the reduction of non-specific binding.

When all of the components outlined herein are used, a preferred method is as follows. The ligated strand is disassociated from the target strand using heat denaturation and incubated under hybridization conditions with a capture probe and a label probe. Generally, this reaction is carried out in the presence of an electrode with at least one immobilized capture probe, although this may also be done in two steps, with the initial incubation and the subsequent addition to the electrode. An optional wash step can be used to remove excess reagents, and detection of the ligated strand proceeds as outlined below.

In one embodiment, a number of capture probes (or capture probes and capture extender probes) that are each substantially complementary to a different portion of the target sequence are used.

As will be appreciated by persons skilled in the art, other methods for detecting the ligated strand are available. For example, other methods can utilize a ligation probe attached to an electrode to which is added a second ligation probe and a target strand. The ligated strand can either be detected directly following disassociation of the target strand, or indirectly following addition of a capture probe comprising a recruitment linker.

The ligated strand can be detected in a variety of ways. In a preferred embodiment, one of the probes comprises at least one covalently attached ETM, and the other probe comprises a sequence that is used to hybridize either directly or indirectly (i.e. through the use of a capture extender probe) to a capture probe on an electrode. For example, the capture probe can hybridize to the second ligation probe (FIG. 8A) or to a first portion of a capture extender probe. The capture extender probe comprises a first portion that hybridizes to the capture probe and a second portion that hybridizes to the second ligation probe (see, e.g., FIG. 14 in WO 00/20476 for a description of capture extender probes; the disclosure of which is incorporated herein by reference in its entirety). Only if both components are present will a signal be generated; this can eliminate the need for removing unligated probes from the system. Alternatively, unligated probes can be removed or washed away, for example using a binding step, etc.

Alternatively, rather than have the probe directly labeled with an ETM, sandwich assay systems can be used (see WO 00/20476 for a description of sandwich assays, the disclosure of which is incorporated herein by reference in its entirety). For example, as shown in FIG. 8A, a sandwich assay comprising capture probe 40 with a sequence complementary to the portion of a ligated strand comprising ligation probe 20 and a label probe 55 comprising at least one ETM and having a sequence complementary to the portion of the ligated strand comprising ligation probe 21 can be used to detect the ligated strand. Other embodiments utilize amplifier probes, label extender probes, etc. as described in WO 00/20476.

If, as illustrated in FIG. 8A, the ligation reaction is done in solution, the ligation complex can be denatured and the ligated strand added to a detection electrode. Preferred embodiments utilize the separation of the ETM label and the capture sequences on different probes. This minimizes or prevents unligated probes comprising ETMs from being captured on the surface. Alternatively, the ligation reaction can be done on a surface, with the capture of the target sequence and then the recruitment of the probe comprising the label (or a probe to which a label probe will bind) to the target sequence (FIG. 8B). Generally, this embodiment utilizes a thermal step to drive off unligated probes, such that only the ligated strands remain on the surface. Similarly, the capture probe itself can be used as a ligation probe, with its terminus comprising the detection position (FIG. 8C). Upon addition of the target sequence and a second ligation probe, a ligation complex can be formed. A label probe (or other probes) can be added as well. Again, this embodiment may require the use of a thermal step to ensure that the target sequence does not remain on the surface unless ligation has occurred.

Accordingly, in a preferred embodiment, the present invention provides arrays, each array location comprising at a minimum a covalently attached nucleic acid probe, such as a capture probe or a ligation probe. By “array locations” or “pads” or “sites” herein meant a location on the substrate that comprises a covalently attached nucleic acid probe. The array locations may comprise electrodes and self-assembled monolayers (SAMs). By “array” herein is meant a plurality of nucleic acid probes in an array format; the size of the array will depend on the composition and end use of the array. Arrays containing from about 2 different capture ligands to many thousands can be made. Generally, the array will comprise from two to as many as 100,000 or more, depending on the size of the electrodes, as well as the end use of the array. Preferred ranges are from about 2 to about 10,000, with from about 5 to about 1000 being preferred, and from about 10 to about 100 being particularly preferred. In some embodiments, the compositions of the invention may not be in array format; that is, for some embodiments, compositions comprising a single capture ligand may be made as well. In addition, in some arrays, multiple substrates may be used, either of different or identical compositions. Thus, for example, large arrays may comprise a plurality of smaller substrates.

In a preferred embodiment, the arrays are present on a substrate. By “substrate” or “solid support” or other grammatical equivalents herein is meant any material that can be modified to contain discrete individual sites appropriate for the attachment or association of nucleic acids. The substrate can comprise a wide variety of materials, as will be appreciated by those in the art, with printed circuit board (PCB) materials being particularly preferred. A description of printed circuit board materials can be found in WO 00/20476 and U.S. Ser. Nos. 09/993,342 and 10,412,660. For a description of other suitable substrates that may be used in the methods and compositions of the present invention, as well as descriptions of the arrays and methods of making them, see WO 00/20476 and U.S. Ser. Nos. 09/993,342 and 10,412,660, the disclosures of which are incorporated herein by reference.

The substrates can comprise electrodes. By “electrode” herein is meant a composition, which, when connected to an electronic device, is able to sense a current or a potential and convert it to a signal. Alternatively an electrode can be defined as a composition which can apply a potential to and/or pass electrons to or from species in the solution. Thus, an electrode is an ETM as described below. Preferred electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste). Preferred electrodes include gold, silicon, platinum, carbon and metal oxide electrodes, with gold being particularly preferred.

The electrodes described herein are depicted as a flat surface, which is only one of the possible conformations of the electrode and is for schematic purposes only. The conformation of the electrode will vary with the detection method used. For example, flat planar electrodes may be preferred for optical detection methods, or when arrays of nucleic acids are made, thus requiring addressable locations for both synthesis and detection. Alternatively, for single probe analysis, the electrode may be in the form of a tube, with the SAMs and probes bound to the inner surface. Electrode coils or mesh may be preferred in some embodiments as well. This allows a maximum of surface area containing the nucleic acids to be exposed to a small volume of sample.

In addition, the detection electrode may be configured to maximize the contact the entire sample has with the electrode, or to allow mixing, etc. See for example WO 00/20476 and U.S. Ser. Nos. 09/993,342 and 10,412,660; the disclosures of which are incorporated herein by reference in their entirety.

Accordingly, in a preferred embodiment, the present invention provides biochips (sometimes referred to herein “chips”) that comprise substrates comprising a plurality of electrodes, preferably gold electrodes. The number of electrodes is as outlined for arrays. Each electrode preferably comprises a self-assembled monolayer (SAM) comprising one or more species (i.e. mixed SAM), including, but not limited to conductive oligomers and/or insulators. See U.S. Publication Nos. 2002/0177135 and 20020121314 for monolayer forming species, methods of making and depositing SAMS on solid substrates, and compositions and methods using SAMs; the disclosures of which are incorporated herein by reference.

In a preferred embodiment, one of the monolayer-forming species comprises a capture probe as described in WO 00/20476, the disclosure of which is incorporated herein by reference in its entirety. In addition, each electrode has an interconnection, that is attached to the electrode at one end and is ultimately attached to a device that can control the electrode. That is, each electrode is independently addressable (see U.S. Publication No. 2002/0177135 and U.S. Ser. No. 10,412,660; the disclosures of which are incorporated herein by reference).

The substrates can be part of a larger device comprising a detection chamber that exposes a given volume of sample to the detection electrode. Generally, the detection chamber ranges from about 1 nL to 1 ml, with about 10 μL to 500 μL being preferred. As will be appreciated by those in the art, depending on the experimental conditions and assay, smaller or larger volumes may be used. See also U.S. Ser. No. 09/295,691, incorporated herein by reference in its entirety.

In some embodiments, the detection chamber and electrode are part of a cartridge that can be placed into a device comprising electronic components (an AC/DC voltage source, an ammeter, a processor, a read-out display, temperature controller, light source, etc.). In this embodiment, the interconnections from each electrode are positioned such that upon insertion of the cartridge into the device, connections between the electrodes and the electronic components are established (see U.S. Publication No. 2002/0177135 and U.S. Serial No. 10,412,660; the disclosures of which are incorporated herein by reference).

Preferably, one of three basic detection mechanisms can be used to detect the ligated strand. In a preferred embodiment, detection relies on the presence of one or more ETMs. Detection of the ETM is based on electron transfer through the stacked π-orbitals of double stranded nucleic acid. This basic mechanism is described in U.S. Pat. Nos. 5,591,578, 5,770,369, 5,705,348, and PCT US97/20014 and is termed “mechanism-1”. Briefly, previous work has shown that electron transfer can proceed rapidly through the stacked π-orbitals of double stranded nucleic acid, and significantly more slowly through single-stranded nucleic acid. Accordingly, this can serve as the basis of an assay. Thus, by adding ETMs (either covalently to one of the strands or non-covalently to the assay complex through the use of hybridization indicators, see U.S. Pat. No. 5,952,172 for a description of hybridization indicators or mediators, the disclosure of which is incorporated herein by reference in its entirety) to a nucleic acid that is attached to a detection electrode via a conductive oligomer, electron transfer between the ETM and the electrode, through the nucleic acid and conductive oligomer, may be detected. See also U.S. Pat. Nos. 6,096,273, 6,221,583, and 6,090,933; the disclosures of which are incorporated herein by reference.

Alternatively, the ETM can be detected, not necessarily via electron transfer through nucleic acid, but rather can be directly detected; that is, the electrons from the ETMs need not travel through the stacked π orbitals in order to generate a signal. This basic idea is termed “mechanism-2”. In this embodiment, the detection electrode preferably comprises a self-assembled monolayer (SAM) that serves to shield the electrode from redox-active species in the sample. The SAM can be formulated to comprise slight “defects” (sometimes referred to herein as “microconduits”, “nanoconduits” or “electroconduits”). Essentially, the electroconduits allow particular ETMs access to the surface. Without being bound by theory, it should be noted that the configuration of the electroconduit depends in part on the ETM chosen. For example, the use of relatively hydrophobic ETMs allows the use of hydrophobic electroconduit forming species, which effectively exclude hydrophilic or charged ETMs. Similarly, the use of more hydrophilic or charged species in the SAM may serve to exclude hydrophobic ETMs. See also, U.S. Publication No. 2002/0177135 and U.S. Serial No. 10,412,660 for a general discussion of electroconduits and methods of making; the disclosures of which are incorporated herein by reference.

Finally, reporterless or labelless systems can also be used to detect SNPs. In these systems, two detection electrodes are used to measure changes in capacitance or impedance as a result of target analyte binding. See generally U.S. Ser. No. 09/458,533, filed Dec. 9, 1999 and PCT US00/33497, the disclosures of which are incorporated herein by reference.

In a preferred embodiment, the detection electrode further comprises a capture probe, preferably covalently attached. By “capture probe” herein is meant a nucleic acid sequence that is used to probe for the presence of the ligated strand, and that binds the ligated strand in a sequence specific manner. In general, for most of the embodiments described herein, at least one capture probe is used per ligated strand.

Generally, the capture probe allows the attachment of the ligated strand or a ligation probe to the detection electrode, for the purposes of detection. Attachment of the ligated stand to the capture probe may be direct (i.e. the ligated probe binds to the capture probe) or indirect (one or more capture extender probes may be used).

As will be appreciated by those in the art, the composition of the capture probe will depend on the composition of the ligated strand or ligation probe. For example, when the ligated strand is a single-stranded nucleic acid, the capture probe is generally a substantially complementary nucleic acid.

Preferred compositions and techniques are outlined in WO 98/20162; PCT/US98/12430; PCT/US98/12082; PCT/US99/01705; PCT/US99/01703; and U.S. Ser. Nos. 09/135,183; 60/105,875; and 09/295,691, for nucleic acid capture probes, all of which are hereby expressly incorporated by reference.

The capture probe can be attached to the electrode via an attachment linker, such as an insulator or conductive oligomer. See WO 00/20476, U.S. Publication No. 20020121314, U.S. Pat. Nos. 6,096,273, 6,221,583, and 6,090,933 for a discussion of means of attaching capture probes to an electrode vian an attachment linker; the disclosures of which are incorporated herein by reference. Other linkers can also be used; for example, homo-or hetero-bifunctional linkers (see 1994 Pierce Chemical Company catalog, technical section on cross-linkers, pages 155-200, incorporated herein by reference). In general, the capture probes are attached to the attachment linker through the use of functional groups on each that can then be used for attachment. Preferred functional groups for attachment are amino groups, carboxy groups, oxo groups and thiol groups.

In a preferred embodiment, there may be one or more different capture probe species on the surface. In some embodiments, there may be one type of capture probe, or one type of capture probe extender. “Capture extender” probes are generally depicted in FIG. 14 of WO 00/20476, and have a first portion that will hybridize to all or part of the capture probe, and a second portion that will hybridize to a portion of ligated strand. Alternatively, different capture probes, or one capture probes with a multiplicity of different capture extender probes can be used. Similarly, it may be desirable (particular in the case of nucleic acid target sequences in mechanism-2 systems) to use auxiliary capture probes that comprise relatively short probe sequences, that can be used to “tack down” components of the system, for example the recruitment linkers, to increase the concentration of ETMs at the surface. See, WO 00/20476, for a description of these embodiments.

In a preferred embodiment, the assay complexes further comprise a label, solution or soluble binding probe, although, for mechanism-1 systems, the ETMs may be added in the form of non-covalently attached hybridization indicators. Solution binding probes are similar to capture probes, in that they bind, preferably specifically, to specific sequences present on the ligated strand. The solution binding probe may be the same or different from the capture probe. Generally, the solution binding probes are not directed attached to the surface, although they may be. The solution binding probe either directly comprises a recruitment linker that comprises at least one ETM, or the recruitment linker binds, either directly or indirectly, to the solution binding probe.

Thus, “solution binding probes” or “soluble binding probes” or “signal carriers” or “label probes” or “label binding probes” with recruitment linkers comprising covalently attached ETMs are provided. That is, one portion of the label probe or solution binding probe directly or indirectly binds to the ligated strand, and one portion comprises a recruitment linker comprising covalently attached ETMs. In some systems, for example in mechanism-1 nucleic acid systems, these may be the same.

The compositions and methods of the invention find use in a variety of applications. In a preferred embodiment, the present invention finds use in detecting target sequences by detecting ligated strands resulting from the chemical ligation of two ligation probes. For example, gene expression analyses may be done, or straight detection of the presence or absence of target sequences. For example, straight detection can be used to detect pathogens and for forensic analysis. In this embodiment, a ligation substrate is formed comprising a target sequence, a first ligation probe with an iodide moiety attached at the 5′ end that is complementary to a first domain on the target sequence and a second ligation probe with at least one ETM and sulfur moiety attached at the 3′ end, complementary to a second domain of the target sequence. That is, the target sequence acts as a “catalyst” orienting the ligation probes in the correct orientation for chemical ligation to occur. If there is no mismatch between the first and second ligation probes and the target sequence, chemical ligation occurs, in which a covalent linkage is formed when the 5′-iodide moiety on the first ligation probe is displaced by the 3′-sulfur moiety on the second ligation. The ligated strand may then be separated from the target sequence via denaturation and hybridized to a capture probe on an electrode for electrochemical detection as described herein.

It should be noted in this context that “mismatch” is a relative term and meant to indicate a difference in the identity of a base at a particular position, termed the “detection position” herein, between two sequences. In general, sequences that differ from wild type sequences are referred to as mismatches. However, particularly in the case of SNPs, what constitutes “wild type” may be difficult to determine as multiple alleles can be relatively frequently observed in the population, and thus “mismatch” in this context requires the artificial adoption of one sequence as a standard. Thus, for the purposes of this invention, sequences are referred to herein as “perfect match” and “mismatch”.

In a preferred embodiment, the present invention finds use in SNP detection and discovery. By “SNP” or “single nucleotide polymorphism” herein is meant a difference or variation, i.e. polymorphism in a single nucleotide. As will be appreciated by those of skill in the art, a SNP may comprise a plurality of nucleotides, either contiguous with each other or separated by one or more nucleotides.

In a preferred embodiment, SNPs are detected using multiple ligation probes (also referred to herein as a ligation probe set). Preferably, each ligation probe set has a different base at the interrogation position and a different covalently attached ETM with a different redox potential. Thus, sets of two probes (for example, when a SNP may exist as one of two different bases), three probes (when an allele comprises 3 different bases) or four probes (to determine the identity of the base at the detection position) can be used. By adding the set of probes to the target sequence and detecting which ETM is present, the identity of the base at the detection position is determined.

In a preferred embodiment, all of the other positions of the probes used in this embodiment are the same; that is, in some embodiments it is preferable to use probes that have all other components equal (e.g. both the length of the probes as well as the non-interrogation bases) to allow good discrimination. This is particularly preferred for SNP discovery and analysis.

Once the assay complexes of the invention are made, the presence of the ETMs at the surface of the monolayer can be detected in a variety of ways. A variety of detection methods may be used, including, but not limited to, optical detection (as a result of spectral changes upon changes in redox states), which includes fluorescence, phosphorescence, luminiscence, chemiluminescence, electrochemiluminescence, and refractive index; and electronic detection, including, but not limited to, amperommetry, voltammetry, capacitance and impedence. These methods include time or frequency dependent methods based on AC or DC currents, pulsed methods, lock-in techniques, filtering (high pass, low pass, band pass), and time-resolved techniques including time-resolved fluorescence.

Detection of electron transfer, i.e. the presence of the ETMs, is generally initiated electronically, with voltage being preferred. Without being limited by the mechanism or theory, detection is based on the transfer of electrons from the ETM to the electrode. Methods of detecting electron transfer are described in WO 00/20476, WO 00/16089 and U.S. Publication Nos. 20030143556, 20020177135, 20020121314, the disclosures of which are hereby incorporated by reference.

In some embodiments, a co-reductant or co-oxidant (collectively, co-redoxant) is used, as an additional electron source or sink. See generally Sato et al., Bull. Chem. Soc. Jpn 66:1032 (1993); Uosaki et al., Electrochimica Acta 36:1799 (1991); and Alleman et al., J. Phys. Chem 100:17050 (1996); all of which are incorporated by reference.

In a preferred embodiment, electronic detection is used, including amperommetry, voltammetry, capacitance, and impedence. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

Alternative electron detection modes also can be utilized. For example, potentiometric (or voltammetric) measurements involve non-faradaic (no net current flow) processes and are utilized traditionally in pH and other ion detectors. Similar sensors are used to monitor electron transfer between the ETM and the electrode. In addition, other properties of insulators (such as resistance) and of conductors (such as conductivity, impedance and capacitance) could be used to monitor electron transfer between ETM and the electrode. Finally, any system that generates a current (such as electron transfer) also generates a small magnetic field, which may be monitored in some embodiments.

In a preferred embodiment, monitoring electron transfer is via amperometric detection. This method of detection involves applying a potential (as compared to a separate reference electrode) between the nucleic acid-conjugated electrode and a reference (counter) electrode in the sample containing target genes of interest. Electron transfer of differing efficiencies is induced in samples in the presence or absence of target nucleic acid; that is, the presence or absence of the target nucleic acid, and thus the label probe, can result in different currents. See WO 00/20476, WO 00/16089 and U.S. Publication Nos. 20030143556, 20020177135, 20020121314, the disclosures of which are hereby incorporated by reference.

In a preferred embodiment, AC initiation and detection methods are used. When AC initiation and detection methods are used, the frequency response of the system changes as a result of the presence of the ETM. By “frequency response” herein is meant a modification of signals as a result of electron transfer between the electrode and the ETM. This modification is different depending on signal frequency. A frequency response includes AC currents at one or more frequencies, phase shifts, DC offset voltages, faradaic impedance, etc. See WO 00/20476, WO 00/16089 and U.S. Publication Nos. 20030143556, 20020177135, 20020121314 for a discussion of AC initiation and detection methods.

The use of combinations of AC and DC initiation signals gives a variety of advantages, including surprising sensitivity and signal maximization. Accordingly, in a preferred embodiment, the first input signal comprises a DC component and an AC component. That is, a DC offset voltage between the sample and counter electrodes is swept through the electrochemical potential of the ETM (for example, when ferrocene is used, the sweep is generally from 0 to 500 mV) (or alternatively, the working electrode is grounded and the reference electrode is swept from 0 to −500 mV). The sweep is used to identify the DC voltage at which the maximum response of the system is seen. This is generally at or about the electrochemical potential of the ETM. Once this voltage is determined, either a sweep or one or more uniform DC offset voltages may be used. DC offset voltages of from about −1 V to about +1.1 V are preferred, with from about −500 mV to about +800 mV being especially preferred, and from about −300 mV to about 500 mV being particularly preferred. In a preferred embodiment, the DC offset voltage is not zero. On top of the DC offset voltage, an AC signal component of variable amplitude and frequency is applied. If the ETM is present, and can respond to the AC perturbation, an AC current will be produced due to electron transfer between the electrode and the ETM. See WO 00/20476, WO 00/16089 and U.S. Publication Nos. 20030143556, 20020177135, 20020121314 for a discussion of AC and DC initiation and detection methods.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Non-Enzymatic Ligation

The efficiency of chemical ligation using phosphate-amine chemical ligation chemistry was analyzed. The ligation assay consisted of: 1) target DNA with two matched probes; and, 2) target DNA with one mismatch.

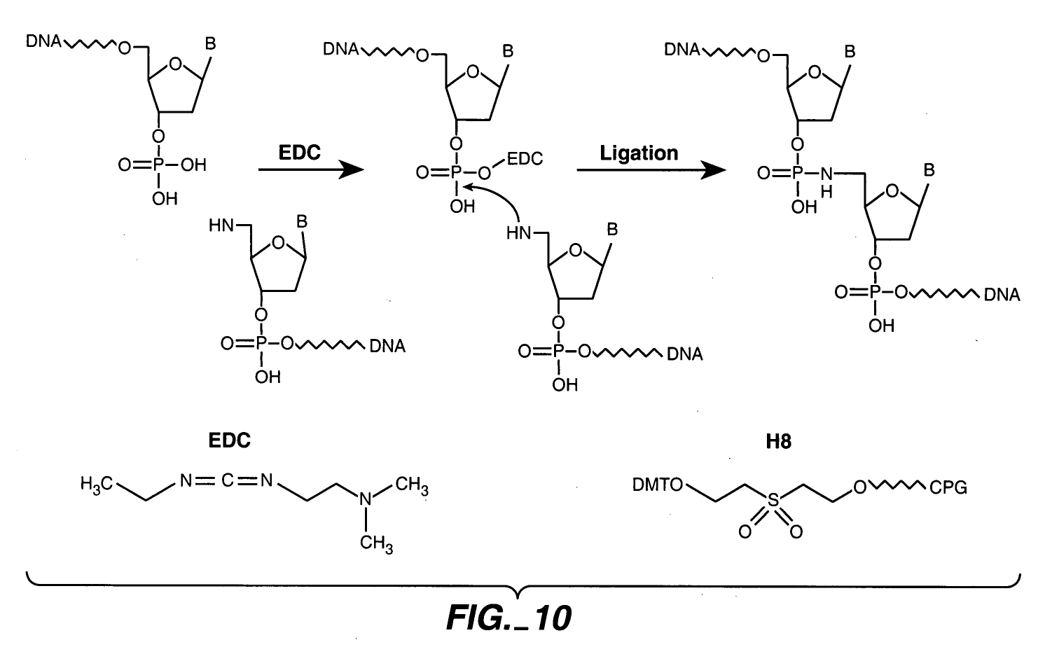

As shown in FIG. 9, the reaction involves a two step process by which 1-ethyl-3-(3-dimethylaminopropyl) carbodiimide (EDC) first binds to a ligation probe labeled with a 3′-phosphate, followed by nucleophillic attack on a 5′-amino group present on a second ligation probe, to form a phosphoramidate bond. The 5′-amino ligation probe was made using Glen Resarch's 5′-amino-dT phosphoramidite. The ligation probe comprising the 3′-phosphate was made using H8 (see FIG. 10).

HPLC analysis was used to detect the peak eluting at 20.3 minutes that corresponded to the ligated strand. In the presence of a complementary target strand, greater than 90% ligation efficiency was observed in approximately 4 hours at 16° C. (FIG. 11A). Greater that 48% efficiency was observed at 2 hours (FIG. 11B) and greater than 95% efficiency was observed with an overnight incubation (FIG. 11C). In contrast, no peak was observed in a complex containing a mismatch following overnight incubation (FIG. 11D).

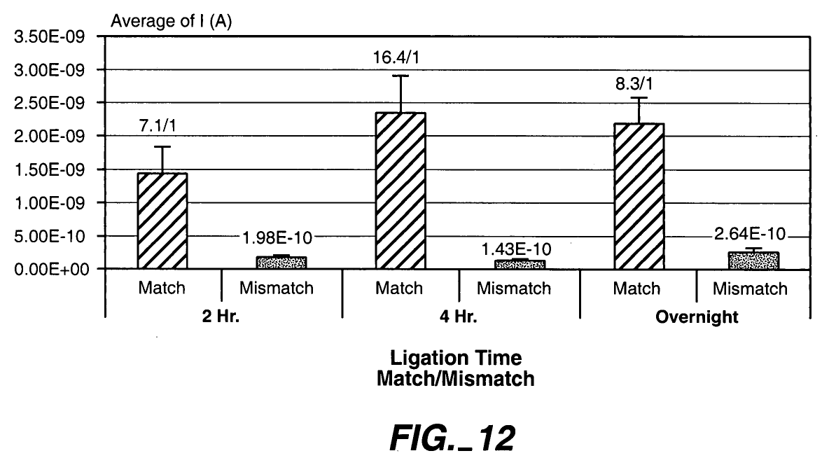

Surface hybridization and electrochemical analysis were preformed to assess the sensitivity of the electrochemical assay to distinguish between “match” and “mismatch”. Samples were taken from the hybridization cocktail at different times and measured electronically. As shown in FIG. 12, the electrochemical assay was capable of distinguishing between a matched complex and a mismatched complex.

1. A composition comprising:
a) a first ligation probe comprising a 5′-iodide moiety; and
b) a second ligation comprising a 3′-sulfur moiety;
wherein at least one of said ligation probes comprises at least a first electron transfer moiety with a first redox potential.

2. A composition according to claim 1 further comprising a target nucleic acid strand with a first domain that hybridizes to said first ligation probe and a second domain that hybridizes to said second ligation probe.

3. A composition according to claim 1 further comprising a third ligation probe comprising a second ETM with a second redox potential different from said first redox potential.

4. A composition according to claim 1 further comprising a fourth ligation probe comprising a third ETM with a third redox potential different from said first or said second redox potential.

5. A composition according to claim 1 further comprising a fifth ligation probe comprising a fourth ETM with a fourth redox potential different from said first, said second or said third redox potential.

6. A composition according to claim 3 wherein said first ligation probe comprises an interrogation position with a first base and said third ligation probe comprises an interrogation position with a second base.

7. A composition according to 5 wherein said first ligation probe comprises an interrogation position with a first base, said third ligation probe comprises an interrogation position with a second base, said fourth ligation probe comprises an interrogation position with a third base, and said fifth ligation probe comprises an interrogation position with a fourth base.

8. A composition according to claim 7 wherein said first, third, fourth and fifth ligation probe comprise the 5′ iodide moiety.

9. A composition according to claim 7 wherein said first, third, fourth and fifth ligation probe comprise the 3′ sulfur moiety.

10. A method of chemical ligation, said method comprising:
a) providing a ligation substrate comprising:
i) a target nucleic acid strand;
ii) a first ligation probe comprising a 5′-iodide moiety; and
iii) a second ligation probe comprising a 3′ sulfur moiety;
wherein at least one of said first and said second ligation probes comprises at least a first electron transfer moiety with a first redox potential;
b) ligating said first and said second ligation probes under conditions wherein said 5′-iodide moiety on said first ligation probe is displaced by said 3′-sulfur moiety on said second ligation probe to form a covalent linkage.

11. A method of detecting a target sequence in a sample comprising:
a) providing a ligation substrate comprising:
i) a target sequence comprising a first domain and an adjacent second domain;
ii) a first ligation probe that is hybridized to said first domain, wherein said first ligation probe comprises a 5′-iodide moiety;
iii) a second ligation probe that is hybridized to said second domain, wherein said second ligation probe comprises a 3′-sulfur moiety;
wherein at least one of said first and second ligation probes comprise at least one electron transfer moiety (ETM);
b) forming a ligation complex comprising said target sequence and a ligated strand by ligating said first and said second ligation probes under conditions wherein said 5′-iodide moiety on said first ligation probe is displaced by said 3′-sulfur moiety on said second ligation probe to form a ligated strand;
c) denaturing said ligation complex;
d) hybridizing said ligated strand to a capture probe on an electrode; and
e) detecting said electron transfer moiety as an indication of the presence of said target sequence in said sample.

12. A method according to claim 11 wherein said first ligation probe comprises said 3′-sulfur moiety and said second ligation probe comprises said 5′-iodide moiety.

13. A method according to claim 11 wherein said ETM is a ferrocene.

14. A method according to claim 11 wherein at least one of said ligation probes comprises a recruitment linker comprising two or more ETMs.

15. A method according to claim 11 wherein said electrode further comprises a self-assembled monolayer.

16. A method of detecting a target sequence in a sample comprising:
a) providing a ligation substrate comprising:
i) a target sequence comprising a first domain and an adjacent second domain;
ii) an electrode comprising a first ligation probe covalently attached to said electrode via an attachment linker, wherein said first ligation probe is hybridized to said first domain of said target sequence; and,
iii) a second ligation probe comprising at least one ETM and wherein said second ligation probe is hybridized to said second domain of said target sequence;
wherein said first ligation probe comprises a 3′-sulfur moiety and said second ligation probe comprises a 5′-iodide moiety;
b) forming a ligation complex comprising said target sequence and a ligated strand by ligating said first and said second ligation probes under conditions wherein said 5′-iodide moiety on said first ligation probe is displaced by said 3′-sulfur moiety on said second ligation probe to form a ligated strand;
c) denaturing said ligation complex, such that said ligated strand comprising said first and said second ligation probes is attached to said electrode; and
d) detecting said electron transfer moiety as an indication of the presence of said target sequence in said sample.

17. A method of detecting a target sequence in a sample comprising:
a) providing a ligation substrate comprising:
i) a target sequence comprising a first domain and an adjacent second domain;
ii) an electrode comprising a first ligation probe covalently attached to said electrode via an attachment linker, wherein said first ligation probe is hybridized to said first domain of said target sequence; and,
iii) a second ligation probe hybridized to said second domain of said target sequence;
wherein said first ligation probe comprises a 3′-sulfur moiety and said second ligation probe comprises a 5′-iodide moiety;
b) forming a ligation complex comprising said target sequence and a ligated strand by ligating said first and said second ligation probes under conditions wherein said 5′-iodide moiety on said first ligation probe is displaced by said 3′-sulfur moiety on said second ligation probe to form a ligated strand;
c) denaturing said ligation complex, such that said ligated strand comprising said first and said second ligation probe is covalently attached to said electrode via said attachment linker;
d) hybridizing said ligated strand to a label probe comprising at least one ETM; and
e) detecting said electron transfer moiety as an indication of the presence of said target sequence in said sample.

18. A method according to claim 16 or 17 wherein said first ligation probe comprises said 5′-idodide moiety and said second ligation probe comprises said 3′-sulfur moiety moiety.

19. A method according to claim 16 or 17 wherein said ETM is a ferrocene.

20. A method according to claim 17 wherein said label probe comprises a recruitment linker comprising two or more ETMs.

21. A method according to claim 16 or 17 wherein said electrode further comprises a self-assembled monolayer.

22. A method of detecting a target sequence in a sample comprising:
a) providing a substrate comprising an array of electrodes each comprising:
i) a self-assembled monolayer (SAM); and,
ii) a different first ligation probe, wherein each of said first ligation probes is capable of hybridizing to a first domain of a target sequence:
iii) a plurality of target sequences comprising a first domain and an adjacent second domain;
iv) a plurality of second ligation probes capable of hybridizing to said second domain of said target sequences;
wherein said first ligation probes comprise a 3′-sulfur moiety and said second ligation probes comprise a 5′-iodide moiety;
b) forming ligation complexes comprising said target sequences and a plurality of ligated strands by ligating said first and said second ligation probes under conditions wherein said 5′-iodide moiety on said first ligation probes displaces said 3′-sulfur moiety on said second ligation probes to form a plurality of ligated strands;
c) denaturing said ligation complexes, such that said ligated strands are covalently attached to said electrodes;
d) hybridizing said ligated strands to a plurality of label probes comprising at least one ETM; and
e) detecting said electron transfer moiety as an indication of the presence of said target sequences in said sample.

23. A method for ligating two probes comprising:
a) providing a ligation substrate comprising:
i) a target sequence comprising a first domain and an adjacent second domain;
ii) a first ligation probe comprising a 3′ OH moiety;
iii) a second ligation probe comprising a 5′ OH moiety;
wherein at least one of said ligation probes comprises at least one covalently attached electron transfer moiety;
b) contacting said ligation substrate with a ligation chemical to ligate said ligation probes to form a ligated strand.

24. A method according to claim 23 wherein said ligation chemical is N-cyanoimidazole.

25. A method according to claim 23 wherein said ligation chemical is 1-ethyl-3-(3-dimethylaminopropyl)carbodiimide hydrochloride (EDC).

26. A method for ligating two probes comprising:
a) providing a ligation substrate comprising:
i) a target sequence comprising a first domain and an adjacent second domain;
ii) a first ligation probe comprising a 3′ phosphate moiety;
iii) a second ligation probe comprising a 5′ amino moiety;
wherein at least one of said ligation probes comprises at least one covalently attached electron transfer moiety;
b) contacting said ligation substrate with a ligation chemical to ligate said ligation probes to form a ligated strand.

27. A method according to claim 26 wherein said ligation chemical is N-cyanoimidazole.

28. A method according to claim 26 wherein said ligation chemical is 1-ethyl-3-(3-dimethylaminopropyl)carbodiimide hydrochloride (EDC).

29. A method for determining the identification of a nucleotide at a detection position in a target sequence comprising a first target domain comprising said detection position and a second target domain adjacent to said detection position, said method comprising:
a) hybridizing a first ligation probe to said first domain, wherein said first ligation probe comprises:
i) a first base at an interrogation position;
ii) a 3′-sulfur moiety; and
iii) a first electron transfer moiety comprising a first redox potential;
b) hybridizing a second ligation probe to said second domain, wherein said second probe comprises a 5′-iodide moiety;
wherein if said first base of said first ligation probe is perfectly complementary to said detection position a ligation substrate is formed;
c) providing conditions wherein said 3′-sulfur moiety on said first ligation probe displaces said 5′-iodide moiety on said second ligation probe to form a ligated strand;
d) forming an assay complex comprising said ligated strand and a capture probe covalently attached to an electrode;
e) detecting the presence or absence of said ETM as an indication of the formation of said ligated strand; and
f) identifying the base at said detection position.

30. A method for determining the identification of a nucleotide at a detection position in a target sequence comprising a first target domain and a second target domain adjacent to said first domain and comprising said detection position, said method comprising:
a) hybridizing a first ligation probe to said first domain, wherein said first ligation probe comprises:
i) a first base at an interrogation position;
ii) a 5′-iodide moiety; and
iii) a first electron transfer moiety comprising a first redox potential;
b) hybridizing a second ligation probe to said second domain, wherein said second probe comprises a 3′-sulfur moiety;
wherein if said first base of said first ligation probe is perfectly complementary to said detection position a ligation substrate is formed;
c) providing conditions wherein said 3′-sulfur moiety on said second ligation probe displaces said 5′-iodide moiety on said first ligation probe to form a ligated strand;
d) forming an assay complex comprising said ligated strand and a capture probe covalently attached to an electrode;
e) detecting the presence or absence of said ETM as an indication of the formation of said ligated strand; and
f) identifying the base at said detection position.
